(19) United States
(12) Patent Application Publication
(10) Pub. No.: US 2021/0145996 A1
(43) Pub. Date: May 20, 2021

(54) Systems and Methods of Sterilizing Parcels, Baggage, and Passenger Screening Divest Trays

(71) Applicant: Rapiscan Systems, Inc., Torrance, CA (US)

(72) Inventors: Edward James Morton, Guildford (GB); Magesh Raman Ramakrishnan, Dorking (GB); Adrian Michael Kelly, Camp Hill (AU); Jeremy Charles Norton, Singapore (SG)

(21) Appl. No.: 17/098,360

(22) Filed: Nov. 14, 2020

Related U.S. Application Data
(60) Provisional application No. 62/936,149, filed on Nov. 15, 2019.

(51) Int. Cl. A61L 2/26; A61L 2/10

(52) U.S. Cl. CPC A61L 2/26; A61L 2202/14; A61L 2202/122; A61L 2/10

(57) ABSTRACT

Screening systems and methods for inspecting and sterilizing objects include a scanner with an upstream side having an entry shroud and a downstream side having an exit shroud. The scanner houses an inspection volume coupled to a radiation source and a detector array. Ultra-violet light curtains are positioned at the entry shroud and a first conveyor transports objects through the ultra-violet light curtains and into the inspection volume for scanning. A second conveyor transports the objects from the inspection volume after scanning.

### CROSS-REFERENCE

The present application relies on, for priority, U.S. Patent Provisional Application No. 62/936,149, titled “Systems and Methods for Handling Passenger Divest Trays at Security Checkpoints”, filed on Nov. 15, 2019, which is herein incorporated by reference in its entirety.

### FIELD

The present specification is related generally to the field of hygienic X-ray screening. More specifically, the present specification is related to the non-contact sterilization of baggage, passenger divest trays, and parcels.

### BACKGROUND

As the world community continues to suffer from the disruption of the COVID-19 pandemic, there remains an on-going need for technology solutions that can either prevent or help mitigate the potential threat imposed by the transmission of harmful pathogens, especially in areas where there is a high concentration of people. Studies have shown that certain viruses, such as the COVID-19 virus, can survive on plastic and metal surfaces for up to 72 hours. Because certain viruses can be transmitted by a human showing little to no symptoms, it is necessary to review potential transmission points and eliminate them where possible. The COVID-19 outbreak is just one example of a global biological threat for which we will need to prepare for, address, and mitigate in the future.

Airports and airlines are already either implementing or investigating the potential use and deployment of thermal camera technology for the monitoring of a passenger's temperature as a means for segregating those with potential illness. While social distancing will still be a consideration or requirement for the travelling public for the unforeseeable future, there is still a risk with physical contact and “close quarter” areas, such as airplanes, which will become the focus in the prevention of virus transmission.

In the context of the aviation security screening industry, hundreds of passengers come into contact daily with each individual plastic tray that is utilized for purpose of transporting carry-on items through the passenger screening X-ray process. This amount of contact, coupled with a virus's potential ability to remain communicable for up to 72 hours on the tray material, poses an extremely high risk. Further, divested passenger items, such as carry-on baggage (which may be placed outside of the tray) and other materials that may be placed into the tray are also high risk items with the potential to carry viral particles.

In addition, for the courier, mailing, and logistics industries, there remains current and continued threat of traces of COVID-19, or other viruses, spreading via parcels that are handled manually at various human touch points. The fear is not lost among logistics workers who constantly wonder if, and when, they will become infected. The time constrained volume flow poses a huge risk to the safety of operations of the logistics industry players.

Accordingly, there is a need to resolve the problem associated with the potential of COVID-19 or other future biological pathogens with high contact transmission rates with divest trays utilized in the passenger screening process by deploying systems and methods for sterilization of each tray after every use. There is also a need to deploy systems and methods to sterilize parcels or packages being handled in courier, mail and logistics industry. The systems and methods of sterilization need to be non-contact, consistently repeatable, and capable of performing sterilization with minimal impact on the operation and throughput of a passenger screening system or a parcel handling/screening system.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a screening system for inspecting an object and sterilizing the object, comprising: a scanner having a housing defining an inspection volume, a radiation source coupled to the inspection volume, and a detector array coupled to the inspection volume, wherein the housing comprises an upstream side configured to allow the object to enter the inspection volume and a downstream side configured to allow the object to exit the inspection volume; an entry shroud having a first end and a second end, wherein the first end is coupled to the upstream side and wherein the entry shroud comprises a first frame extending between the first end and the second end; an exit shroud having a third end and a fourth end, wherein the third end is coupled to the downstream side and wherein the exit shroud comprises a second frame extending between the third end and the fourth end; a first plurality of ultraviolet light curtains coupled to the entry shroud, wherein the first plurality of ultraviolet light curtains are positioned between the first end and the second end; and a first conveyor extending at least from the second end to the first end of the entry shroud and a second conveyor extending at least from the third end to the fourth end of the exit shroud, wherein the first conveyor is configured to transport the object from the second end through the first plurality of ultra-violet light curtains to the inspection volume and the second conveyor is configured to transport the object from the inspection volume after scanning to the fourth end.

Optionally, each of the first conveyor and the second conveyor includes at least two belts and wherein the at least two belts having at least one split space between them.

Optionally, each of the first plurality of ultra-violet light curtains comprises a first ultra-violet light positioned along a first side of the first frame, a second ultra-violet light positioned along a second side of the first frame, wherein the second side opposes the first side, a third ultra-violet light positioned along a third side of the first frame and a fourth ultra-violet light positioned along a fourth side of the first frame, wherein the third side opposes the fourth side. Optionally, the first ultra-violet light, the second ultra-violet light and the third ultra-violet light are positioned above belt surfaces of the first conveyor and are configured to illuminate a first surface, a second surface and a third surface, respectively, of the object while being transported by the first conveyor. Optionally, the fourth ultra-violet light is positioned below the belt surfaces to direct ultra-violet light through a space in the belt surfaces to thereby illuminate a fourth surface of the object.

Optionally, each of the first plurality of ultra-violet light curtains comprises a first ultra-violet light source positioned along a first side of the first frame, a second ultra-violet light source positioned along a second side of the first frame, wherein the second side opposes the first side, a third ultra-violet light source positioned along a third side of the first frame and a fourth ultra-violet light source positioned along a fourth side of the first frame, wherein the third side opposes the fourth side. Optionally, the first ultra-violet light source, the second ultra-violet light source and the third ultra-violet light source are positioned above belt surfaces of the first conveyor and are configured to illuminate a first surface, a second surface and a third surface, respectively, of the object while being transported by the first conveyor. Optionally, the fourth ultra-violet light source is positioned below the belt surfaces to direct ultra-violet light through a space in the belt surfaces to thereby illuminate a fourth surface of the object.

Optionally, each of the first plurality of ultra-violet light curtains comprises at least one first ultra-violet light source positioned along a first side of the first frame, at least one second ultra-violet light source positioned along a second side of the first frame, wherein the second side opposes the first side, at least one third ultra-violet light source positioned along a third side of the first frame and at least one fourth ultra-violet light source positioned along a fourth side of the first frame, wherein the third side opposes the fourth side. Optionally, the at least one first ultra-violet light source, at least one second ultra-violet light source and at least one third ultra-violet light source are positioned above belt surfaces of the first conveyor and are configured to illuminate a first surface, a second surface and a third surface, respectively, of the object while being transported by the first conveyor. Optionally, the at least one fourth ultra-violet light source is positioned below the belt surfaces to direct ultra-violet light through a space in the belt surfaces to thereby illuminate a fourth surface of the object.

Optionally, first frame lies in a plane that is substantially orthogonal to a direction of movement of the object by the first conveyor.

Optionally, each of the first plurality of ultra-violet light curtains uses light sources emitting ultra-violet radiation in a wavelength range of 280-100 nm or photon energy in a range of 4.43-12.4 eV.

Optionally, the scanning system further comprises a lead lined cover coupled to at least one of the first frame and the second frame.

Optionally, the entry shroud comprises a first portion extending from the second end, wherein the first portion is coupled to a second portion, wherein the second portion is coupled to a third portion, wherein the third portion is coupled to the first end, and wherein the first portion and third portion are positioned at a right angle to each other.

Optionally, the entry shroud comprises a first portion extending from the second end, wherein the first portion is coupled to a second portion, wherein the second portion is coupled to a third portion, wherein the third portion is coupled to the first end, wherein a longitudinal axis of the first portion and a longitudinal axis of the third portion are parallel to each other, and wherein the second portion is skewed at an angle of 45 degrees relative to the longitudinal axes of both the first portion and the third portion.

Optionally, the entry shroud comprises a first portion extending from the second end, wherein the first portion is coupled to a second portion, wherein the second portion is coupled to a third portion, wherein the third portion is coupled to the first end, and wherein the first portion and third portion are configured such that a longitudinal axis of the first portion and a longitudinal axis of the third portion are neither parallel nor orthogonal to each other.

Optionally, the object is a parcel or a baggage.

The present specification also discloses a method of sterilizing an item to be scanned by a scanning system having an upstream side and a downstream side adapted to allow the item to enter and exit, respectively, the scanning system, wherein the upstream side is coupled to an entry shroud having a first end and a second end, wherein the downstream side is coupled to an exit shroud having a third end and a fourth end, wherein a first plurality of ultra-violet light curtains are positioned within the entry shroud between the first end and the second end, the method comprising: placing the item on a first conveyor at the second end, wherein the first conveyor extends at least from the second end to the first end within the entry shroud; transporting, using the first conveyor, the item from the second end to the scanning system through said first plurality of ultra-violet light curtains, wherein each of said first plurality of ultra-violet light curtains has a least a first ultra-violet light source positioned along a first side, at least a second ultra-violet light source positioned along a second side opposing the first side, at least a third ultra-violet light source positioned along a third side and at least a fourth ultra-violet light source positioned along a fourth side opposing the third side; and illuminating first, second, third and fourth surfaces of the item by the at least one first, at least one second, at least one third and at least one fourth ultra-violet light sources while the item is being transported by the first conveyor.

Optionally, the method further comprises: scanning the item using the scanning system; and transporting, using a second conveyor, the item from the scanning system to said fourth end, wherein said second conveyor extends at least from the third end to the fourth end within the exit shroud. A second plurality of ultra-violet light curtains may be positioned within the exit shroud between the third end and the fourth end. Each of the second plurality of ultra-violet light curtains may have at least one first ultra-violet light source positioned along a first side, at least one second ultra-violet light source positioned along a second side opposing the first side, at least one third ultra-violet light source positioned along a third side and at least one fourth ultra-violet light source positioned along a fourth side opposing the third side. Optionally, the method further comprises: illuminating first, second, third and fourth surfaces of the item by the at least one first, at least one second, at least one third and at least one fourth ultra-violet light source of each of said second plurality of ultra-violet light curtains while the item is being transported by the second conveyor.

The present specification also discloses a system for sterilizing a divest tray, comprising: a screening system having entry and exit areas; a first conveyor configured to transport the divest tray from a divest area to the screening system for scanning; a second conveyor configured to transport the divest tray, cleared after scanning, from the screening system to a reclaim area, wherein the second conveyor is positioned at a first level; an elevator positioned beyond the reclaim area and configured to transfer the divest tray from the second conveyor onto a third conveyor, wherein the third conveyor has a plurality of rollers and is positioned at a second level below the first level, and wherein the plurality of rollers have spaces in between; and a plurality of ultra-violet light curtains positioned in said spaces and along at least a portion of said third conveyor, wherein said third conveyor is configured to transport the divest tray back towards the divest area through the plurality of ultra-violet light curtains.

Optionally, each of said plurality of ultra-violet light curtains has at least one first ultra-violet light source positioned along a first side, at least one second ultra-violet light source positioned along a second side opposing the first side, at least one third ultra-violet light source positioned along a third side and at least one fourth ultra-violet light source positioned along a fourth side opposing the third side. Optionally, the at least one first, at least one second and at least one third ultra-violet light sources are positioned above the surfaces of the rollers and respectively illuminate first, second and third surfaces of the divest tray while being transported by the third conveyor, and wherein the at least one fourth ultra-violet light source transmits ultra-violet light through said spaces to illuminate a fourth surface of the divest tray.

Optionally, a plane of each of said plurality of ultra-violet light curtains lies substantially orthogonal to a direction of movement of the divest tray by the third conveyor. Optionally, each of said plurality of ultra-violet light curtains uses light sources emitting ultra-violet radiation in a wavelength range of 280-100 nm or photon energy range of 4.43-12.4 eV. Optionally, the portion of said third conveyor layer is enclosed within a housing to prevent ultra-violet light exposure to personnel and/or passengers in the vicinity of the portion. The portion may extend from the elevator to the exit area of the screening system. The portion may extend from the elevator to the entry area of the screening system.

The present specification also discloses a method of sterilizing a divest tray, the method comprising: transferring the divest tray from a divest area onto a first conveyor; transporting, using the first conveyor, the divest tray from the divest area to the screening system for scanning one or more items placed in the divest tray at the divest area; scanning, using the screening system, the one or more items; transporting, using a second conveyor, the divest tray, cleared after scanning, from the scanning system to a reclaim area, wherein the second conveyor is positioned at a first level, and wherein the one or more items are reclaimed at the reclaim area; transferring, using an elevator, the divest tray from the second conveyor onto a third conveyor, wherein the third conveyor has a plurality of rollers and is positioned at a second level below the first level, wherein the plurality of rollers have spaces in between, and wherein a plurality of ultra-violet light curtains are positioned in said spaces and along at least a portion of the third conveyor; and transporting, using the third conveyor, the divest tray from the elevator back to the divest area through the plurality of ultra-violet light curtains.

Optionally, each of said plurality of ultra-violet light curtains has a first plurality of ultra-violet light sources positioned along a first side, a second plurality of ultra-violet light sources positioned along a second side opposing the first side, a third plurality of ultra-violet light sources positioned along a third side and a fourth plurality of ultra-violet light sources positioned along a fourth side opposing the third side. Optionally, the first, second and third plurality of ultra-violet light sources are positioned above the surfaces of the rollers and respectively illuminate first, second and third surfaces of the divest tray while being transported by the third conveyor, and wherein the fourth plurality of ultra-violet light sources transmits ultra-violet light through said spaces to illuminate a fourth surface of the divest tray.

Optionally, each of said plurality of ultra-violet light curtains has a first ultra-violet light source positioned along a first side, a second ultra-violet light source positioned along a second side opposing the first side, a third ultra-violet light source positioned along a third side and a fourth ultra-violet light source positioned along a fourth side opposing the third side, and wherein the first, second and third ultra-violet light sources are positioned above the surfaces of the rollers and respectively illuminate first, second and third surfaces of the divest tray while being transported by the third conveyor, and wherein the fourth ultra-violet light source transmits ultra-violet light through said spaces to illuminate a fourth surface of the divest tray.

Optionally, a plane of each of said plurality of ultra-violet light curtains lies substantially orthogonal to a direction of movement of the divest tray by the third conveyor.

Optionally, each of said plurality of ultra-violet light curtains uses light sources emitting ultra-violet radiation in a wavelength range of 280-100 nm or photon energy range of 4.43-12.4 eV.

Optionally, the portion of said third conveyor layer is enclosed within a housing to prevent ultra-violet light exposure to personnel and/or passengers in the vicinity of the portion. The portion may extend from the elevator to the exit area of the screening system. The portion may extend from the elevator to the entry area of the screening system.

The present specification also discloses a system for sterilizing a divest tray, comprising: a screening system having entry and exit areas; a first conveyor configured to transport the divest tray from a divest area to the screening system for scanning; a second conveyor configured to transport the divest tray, cleared after scanning, from the screening system to a reclaim area, wherein the second conveyor is positioned at a first level; an elevator positioned beyond the reclaim area and configured to transfer the divest tray from the second conveyor onto a third conveyor, wherein the third conveyor has a plurality of rollers and is positioned at a second level below the first level, wherein the plurality of rollers have spaces in between, and wherein the third conveyor is configured to transport the divest tray back towards the divest area; a first plurality of nozzles positioned vertically along the elevator, said first plurality of nozzles being configured to spray a disinfectant onto the divest tray while the divest tray is being transferred by the elevator; a second plurality of nozzles positioned in said spaces, said second plurality of nozzles being configured to spray the disinfectant onto a base of the divest tray while the divest tray is being transported by the third conveyor; a plurality of rotating brushes positioned along a first portion of the third conveyor to clean at least a top surface of the divest tray while the divest tray is being transported by the third conveyor; and a drying system positioned along a second portion of the third conveyor to remove the disinfectant and dry the divest tray while the divest tray is being transported by the third conveyor.

Optionally, the first portion extends from the elevator to the exit area of the screening system. Optionally, the second portion lays beyond the plurality of rotating brushes and prior to the divest area.

The drying system may include at least one of dry rotating brushes, infra-red lamps, or hot air jets.

Optionally, the system further comprises: a first plurality of ultra-violet light curtains positioned in said spaces and in said first portion, wherein each of said first plurality of ultra-violet light curtains has a first ultra-violet light source positioned along a first side, a second ultra-violet light source positioned along a second side opposing the first side, a third ultra-violet light source positioned along a third side and a fourth ultra-violet light source positioned along a fourth side opposing the third side, and wherein the first, second and third ultra-violet light sources are positioned above the surfaces of the rollers and respectively illuminate first, second and third surfaces of the divest tray while being transported by the third conveyor, and wherein the fourth ultra-violet light source transmits ultra-violet light through said spaces to illuminate a fourth surface of the divest tray. Optionally, a plane of each of said first plurality of ultra-violet light curtains lies substantially orthogonal to a direction of movement of the divest tray by the third conveyor.

Optionally, the system further comprises a first plurality of ultra-violet light curtains positioned in said spaces and in said first portion, wherein each of said first plurality of ultra-violet light curtains has a first ultra-violet light source positioned along a first side, a second ultra-violet light source positioned along a second side opposing the first side, a third ultra-violet light source positioned along a third side and a fourth ultra-violet light source positioned along a fourth side opposing the third side, and wherein the first, second and third ultra-violet light sources are positioned above the surfaces of the rollers and respectively illuminate first, second and third surfaces of the divest tray while being transported by the third conveyor, and wherein the fourth ultra-violet light source transmits ultra-violet light through said spaces to illuminate a fourth surface of the divest tray.

Optionally, the system further comprises: a second plurality of ultra-violet light curtains positioned in said spaces beyond said drying system and prior to the divest area, wherein each of said second plurality of ultra-violet light curtains has a first ultra-violet light source positioned along a first side, a second ultra-violet light source positioned along a second side opposing the first side, a third ultra-violet light source positioned along a third side and a fourth ultra-violet light source positioned along a fourth side opposing the third side, and wherein the first, second and third ultra-violet light sources are positioned above the surfaces of the rollers and respectively illuminate first, second and third surfaces of the divest tray while being transported by the third conveyor, and wherein the fourth ultra-violet light source transmits ultra-violet light through said spaces to illuminate a fourth surface of the divest tray.

Optionally, the system further comprises: a second plurality of ultra-violet light curtains positioned in said spaces beyond said drying system and prior to the divest area, wherein each of said second plurality of ultra-violet light curtains has a first plurality of ultra-violet lights positioned along a first side, a second plurality of ultra-violet lights positioned along a second side opposing the first side, a third plurality of ultra-violet lights positioned along a third side and a fourth plurality of ultra-violet lights positioned along a fourth side opposing the third side, and wherein the first, second and third plurality of ultra-violet lights are positioned above the surfaces of the rollers and respectively illuminate first, second and third surfaces of the divest tray while being transported by the third conveyor, and wherein the fourth plurality of ultra-violet lights beam ultra-violet light through said spaces to illuminate a fourth surface of the divest tray. Optionally, a plane of each of said second plurality of ultra-violet light curtains lies substantially orthogonal to a direction of movement of the divest tray by the third conveyor. Optionally, each of said first and second plurality of ultra-violet light curtains uses light sources emitting ultra-violet radiation in a wavelength range of 280-100 nm or photon energy range of 4.43-12.4 eV.

The present specification also discloses a method of sterilizing a divest tray, the method comprising: transferring the divest tray from a divest area onto a first conveyor; transporting, using the first conveyor, the divest tray from the divest area to a screening system for scanning one or more items placed in the divest tray at the divest area; scanning, using the screening system, the one or more items; transporting, using a second conveyor, the divest tray, cleared after scanning, from the scanning system to a reclaim area, wherein the second conveyor is positioned at a first level, and wherein the one or more items are reclaimed at the reclaim area; transferring, using an elevator positioned beyond the reclaim area, the divest tray from the second conveyor onto a third conveyor, wherein the third conveyor has a plurality of rollers and is positioned at a second level below the first level, and wherein the plurality of rollers have spaces in between; spraying, using a first plurality of nozzles positioned vertically along the elevator, a disinfectant onto the divest tray while the divest tray is being transferred by the elevator; transporting, using the third conveyor, the divest tray from the elevator back to the divest area; spraying, using a second plurality of nozzles positioned in said spaces, the disinfectant onto a base of the divest tray while the divest tray is being transported by the third conveyor; cleaning, using a plurality of rotating brushes positioned along a first portion of the third conveyor, at least a top surface of the divest tray while the divest tray is being transported by the third conveyor; and removing the disinfectant and drying the divest tray, using a drying system positioned along a second portion of the third conveyor, while the divest tray is being transported by the third conveyor.

The first portion may extend from the elevator to the exit area of the screening system. The second portion may lay beyond the plurality of rotating brushes and prior to the divest area.

The drying system may include at least one of dry rotating brushes, infra-red lamps, or hot air jets. Optionally, the method further comprises: illuminating, using a first plurality of ultra-violet light curtains positioned in said spaces and in said first portion, the divest tray being transported by the third conveyor, wherein each of said first plurality of ultra-violet light curtains has a first ultra-violet light source positioned along a first side, a second ultra-violet light source positioned along a second side opposing the first side, a third ultra-violet light source positioned along a third side and a fourth ultra-violet light source positioned along a fourth side opposing the third side, and wherein the first, second and third ultra-violet light sources are positioned above the surfaces of the rollers and respectively illuminate first, second and third surfaces of the divest tray while being transported by the third conveyor, and wherein the fourth ultra-violet light source transmits ultra-violet light through said spaces to illuminate a fourth surface of the divest tray.

Optionally, the method further comprises: illuminating, using a first plurality of ultra-violet light curtains positioned in said spaces and in said first portion, the divest tray being transported by the third conveyor, wherein each of said first plurality of ultra-violet light curtains has a first plurality of ultra-violet lights positioned along a first side, a second plurality of ultra-violet lights positioned along a second side opposing the first side, a third plurality of ultra-violet lights positioned along a third side and a fourth plurality of ultra-violet lights positioned along a fourth side opposing the third side, and wherein the first, second and third plurality of ultra-violet lights are positioned above the surfaces of the rollers and respectively illuminate first, second and third surfaces of the divest tray, and wherein the fourth plurality of ultra-violet lights beam ultra-violet light through said spaces to illuminate a fourth surface of the divest tray. Optionally, a plane of each of said first plurality of ultra-violet light curtains lies substantially orthogonal to a direction of movement of the divest tray by the third conveyor.

Optionally, the method further comprises: illuminating, using a second plurality of ultra-violet light curtains positioned in said spaces beyond said drying system and prior to the divest area, the divest tray being transported by the third conveyor, wherein each of said second plurality of ultra-violet light curtains has a first ultra-violet light source positioned along a first side, a second ultra-violet light source positioned along a second side opposing the first side, a third ultra-violet light source positioned along a third side and a fourth ultra-violet light source positioned along a fourth side opposing the third side, and wherein the first, second and third ultra-violet light sources are positioned above the surfaces of the rollers and respectively illuminate first, second and third surfaces of the divest tray while being transported by the third conveyor, and wherein the fourth ultra-violet light source transmits ultra-violet light through said spaces to illuminate a fourth surface of the divest tray.

Optionally, the method further comprises: illuminating, using a second plurality of ultra-violet light curtains positioned in said spaces beyond said drying system and prior to the divest area, the divest tray being transported by the third conveyor, wherein each of said second plurality of ultra-violet light curtains has a first plurality of ultra-violet lights positioned along a first side, a second plurality of ultra-violet lights positioned along a second side opposing the first side, a third plurality of ultra-violet lights positioned along a third side and a fourth plurality of ultra-violet lights positioned along a fourth side opposing the third side, and wherein the first, second and third plurality of ultra-violet lights are positioned above the surfaces of the rollers and respectively illuminate first, second and third surfaces of the divest tray, and wherein the fourth plurality of ultra-violet lights beam ultra-violet light through said spaces to illuminate a fourth surface of the divest tray.

Optionally, a plane of each of said second plurality of ultra-violet light curtains lies substantially orthogonal to a direction of movement of the divest tray by the third conveyor.

Optionally, each of said first and second plurality of ultra-violet light curtains uses light sources emitting ultra-violet radiation in a wavelength range of 280-100 nm or photon energy range of 4.43-12.4 eV.

The present specification also discloses a system for sterilizing a divest tray, comprising: a first conveyor configured to transport the divest tray from a divest area to a screening system, wherein said screening system has an inspection tunnel extending between an entrance and an exit, a fourth conveyor having an associated first belt configured to receive the divest tray from the first conveyor and transport the divest tray in a first direction through the inspection tunnel for scanning, wherein said first belt has a first plurality of flaps spaced apart from each other by a predefined distance, and wherein each of said first plurality of flaps is hinged up from a surface of said first belt behind a trailing edge of the divest tray as the divest tray is received on said first belt; a second conveyor configured to transport the divest tray, cleared after scanning, from the screening system to a reclaim area, wherein the second conveyor is positioned at a first level; an elevator positioned beyond the reclaim area and configured to transfer the divest tray from the second conveyor onto a third conveyor, wherein the third conveyor has a plurality of rollers and is positioned at a second level below the first level, wherein the plurality of rollers have spaces in between, and wherein the third conveyor is configured to transport the divest tray back towards the divest area; a first plurality of nozzles positioned vertically along the elevator, said first plurality of nozzles being configured to spray a disinfectant onto the divest tray while the divest tray is being transferred by the elevator; a second plurality of nozzles positioned in said spaces, said second plurality of nozzles being configured to spray the disinfectant onto a base of the divest tray while the divest tray is being transported by the third conveyor; a plurality of rotating brushes positioned along a first portion of the third conveyor to clean at least a top surface of the divest tray while the divest tray is being transported by the third conveyor; and a drying system positioned along a second portion of the third conveyor to remove the disinfectant and dry the divest tray while the divest tray is being transported by the third conveyor.

Optionally, each of said first plurality of flaps is configured to catch the divest tray as it passes through one or more shielding curtains at the entrance.

Optionally, each of the first plurality of flaps folds back down to lay flush with the surface as said first belt loops below the fourth conveyor in a second direction opposite to the first direction. Each of the first plurality of flaps may be hinged up using a mechanical lever and an actuator as said first belt loops above the fourth conveyor in said first direction.

Optionally, the third conveyor has an associated second belt, and said second belt has a second plurality of flaps spaced apart from each other by a predefined distance, wherein each of said second plurality of flaps is hinged up from a surface of said second belt to prevent the divest tray from dislodging while being cleaned by the plurality of rotating brushes or being dried by the drying system.

Optionally, the first portion extends from the elevator to the exit of the screening system. Optionally, the second portion lays beyond the plurality of rotating brushes and prior to the divest area.

The drying system may include at least one of dry rotating brushes, infra-red lamps, or hot air jets.

Optionally, the system further comprises: a first plurality of ultra-violet light curtains positioned in said spaces and in said first portion, wherein each of said first plurality of ultra-violet light curtains has a first ultra-violet light source positioned along a first side, a second ultra-violet light source positioned along a second side opposing the first side, a third ultra-violet light source positioned along a third side and a fourth ultra-violet light source positioned along a fourth side opposing the third side, and wherein the first, second and third ultra-violet light sources are positioned above the surfaces of the rollers and respectively illuminate first, second and third surfaces of the divest tray while being transported by the third conveyor, and wherein the fourth ultra-violet light source transmits ultra-violet light through said spaces to illuminate a fourth surface of the divest tray.

Optionally, the system further comprises: a first plurality of ultra-violet light curtains positioned in said spaces and in said first portion, wherein each of said first plurality of ultra-violet light curtains has a first plurality of ultra-violet lights positioned along a first side, a second plurality of ultra-violet lights positioned along a second side opposing the first side, a third plurality of ultra-violet lights positioned along a third side and a fourth plurality of ultra-violet lights positioned along a fourth side opposing the third side, and wherein the first, second and third plurality of ultra-violet lights are positioned above the surfaces of the rollers and respectively illuminate first, second and third surfaces of the divest tray while being transported by the third conveyor, and wherein the fourth plurality of ultra-violet lights beam ultra-violet light through said spaces to illuminate a fourth surface of the divest tray.

Optionally, a plane of each of said first plurality of ultra-violet light curtains lies substantially orthogonal to a direction of movement of the divest tray by the third conveyor.

Optionally, the system further comprises: a second plurality of ultra-violet light curtains positioned in said spaces beyond said drying system and prior to the divest area, wherein each of said second plurality of ultra-violet light curtains has a first ultra-violet light source positioned along a first side, a second ultra-violet light source positioned along a second side opposing the first side, a third ultra-violet light source positioned along a third side and a fourth ultra-violet light source positioned along a fourth side opposing the third side, and wherein the first, second and third ultra-violet light sources are positioned above the surfaces of the rollers and respectively illuminate first, second and third surfaces of the divest tray while being transported by the third conveyor, and wherein the fourth ultra-violet light source transmits ultra-violet light through said spaces to illuminate a fourth surface of the divest tray.

Optionally, the system further comprises: a second plurality of ultra-violet light curtains positioned in said spaces beyond said drying system and prior to the divest area, wherein each of said second plurality of ultra-violet light curtains has a first plurality of ultra-violet lights positioned along a first side, a second plurality of ultra-violet lights positioned along a second side opposing the first side, a third plurality of ultra-violet lights positioned along a third side and a fourth plurality of ultra-violet lights positioned along a fourth side opposing the third side, and wherein the first, second and third plurality of ultra-violet lights are positioned above the surfaces of the rollers and respectively illuminate first, second and third surfaces of the divest tray while being transported by the third conveyor, and wherein the fourth plurality of ultra-violet lights beam ultra-violet light through said spaces to illuminate a fourth surface of the divest tray.

Optionally, a plane of each of said second plurality of ultra-violet light curtains lies substantially orthogonal to a direction of movement of the divest tray by the third conveyor.

Optionally, each of said first and second plurality of ultra-violet light curtains uses light sources emitting ultra-violet radiation in a wavelength range of 280-100 nm or photon energy range of 4.43-12.4 eV.

The present specification also discloses a method of sterilizing a divest tray, the method comprising: transferring the divest tray from a divest area onto a first conveyor; transporting, using the first conveyor, the divest tray from the divest area to a screening system for scanning one or more items placed in the divest tray at the divest area, wherein said screening system has an inspection tunnel extending between an entrance and an exit; receiving, onto a fourth conveyor having an associated first belt moving in a first direction, the divest tray from the first conveyor, wherein said first belt has a first plurality of flaps spaced apart from each other by a predefined distance; hinging upward, using a mechanical lever and an actuator, each of said first plurality of flaps from a surface of said first belt behind a trailing edge of the divest tray as the divest tray is received onto the fourth conveyor; transporting, using the fourth conveyor, the divest tray in said first direction through the inspection tunnel; transporting, using a second conveyor, the divest tray, cleared after scanning, from the scanning system to a reclaim area, wherein the second conveyor is positioned at a first level, and wherein the one or more items are reclaimed at the reclaim area; transferring, using an elevator positioned beyond the reclaim area, the divest tray from the second conveyor onto a third conveyor, wherein the third conveyor has a plurality of rollers and is positioned at a second level below the first level, and wherein the plurality of rollers have spaces in between; spraying, using a first plurality of nozzles positioned vertically along the elevator, a disinfectant onto the divest tray while the divest tray is being transferred by the elevator; transporting, using the third conveyor, the divest tray from the elevator back to the divest area; spraying, using a second plurality of nozzles positioned in said spaces, the disinfectant onto a base of the divest tray while the divest tray is being transported by the third conveyor; cleaning, using a plurality of rotating brushes positioned along a first portion of the third conveyor, at least a top surface of the divest tray while the divest tray is being transported by the third conveyor; and removing the disinfectant and drying the divest tray, using a drying system positioned along a second portion of the third conveyor, while the divest tray is being transported by the third conveyor.

Optionally, each of said first plurality of flaps is configured to catch the divest tray as it passes through one or more shielding curtains at the entrance.

Optionally, each of the first plurality of flaps folds back down to lay flush with the surface as said first belt loops below the fourth conveyor in a second direction opposite to the first direction.

Optionally, the third conveyor has an associated second belt, and said second belt has a second plurality of flaps spaced apart from each other by a predefined distance, wherein each of said second plurality of flaps is hinged up from a surface of said second belt to prevent the divest tray from dislodging while being cleaned by the plurality of rotating brushes or being dried by the drying system.

Optionally, the first portion extends from the elevator to the exit of the screening system. Optionally, the second portion lays beyond the plurality of rotating brushes and prior to the divest area.

Optionally, the drying system includes at least one of dry rotating brushes, infra-red lamps, or hot air jets.

Optionally, a first plurality of ultra-violet light curtains are positioned in said spaces and in said first portion, wherein each of said first plurality of ultra-violet light curtains has a first ultra-violet light source positioned along a first side, a second ultra-violet light source positioned along a second side opposing the first side, a third ultra-violet light source positioned along a third side and a fourth ultra-violet light source positioned along a fourth side opposing the third side, and wherein the first, second and third ultra-violet light sources are positioned above the surfaces of the rollers and respectively illuminate first, second and third surfaces of the divest tray while being transported by the third conveyor, and wherein the fourth ultra-violet light source transmits ultra-violet light through said spaces to illuminate a fourth surface of the divest tray.

Optionally, a first plurality of ultra-violet light curtains are positioned in said spaces and in said first portion, wherein each of said first plurality of ultra-violet light curtains has a first plurality of ultra-violet lights positioned along a first side, a second plurality of ultra-violet lights positioned along a second side opposing the first side, a third plurality of ultra-violet lights positioned along a third side and a fourth plurality of ultra-violet lights positioned along a fourth side opposing the third side, and wherein the first, second and third plurality of ultra-violet lights are positioned above the surfaces of the rollers and respectively illuminate first, second and third surfaces of the divest tray while being transported by the third conveyor, and wherein the fourth plurality of ultra-violet lights beam ultra-violet light through said spaces to illuminate a fourth surface of the divest tray.

Optionally, a plane of each of said first plurality of ultra-violet light curtains lies substantially orthogonal to a direction of movement of the divest tray by the third conveyor.

Optionally, a second plurality of ultra-violet light curtains is positioned in said spaces beyond said drying system and prior to the divest area, wherein each of said second plurality of ultra-violet light curtains has a first ultra-violet light source positioned along a first side, a second ultra-violet light source positioned along a second side opposing the first side, a third ultra-violet light source positioned along a third side and a fourth ultra-violet light source positioned along a fourth side opposing the third side, and wherein the first, second and third ultra-violet light sources are positioned above the surfaces of the rollers and respectively illuminate first, second and third surfaces of the divest tray while being transported by the third conveyor, and wherein the fourth ultra-violet light source transmits ultra-violet light through said spaces to illuminate a fourth surface of the divest tray.

Optionally, a second plurality of ultra-violet light curtains is positioned in said spaces beyond said drying system and prior to the divest area, wherein each of said second plurality of ultra-violet light curtains has a first plurality of ultra-violet lights positioned along a first side, a second plurality of ultra-violet lights positioned along a second side opposing the first side, a third plurality of ultra-violet lights positioned along a third side and a fourth plurality of ultra-violet lights positioned along a fourth side opposing the third side, and wherein the first, second and third plurality of ultra-violet lights are positioned above the surfaces of the rollers and respectively illuminate first, second and third surfaces of the divest tray while being transported by the third conveyor, and wherein the fourth plurality of ultra-violet lights beam ultra-violet light through said spaces to illuminate a fourth surface of the divest tray. Optionally, a plane of each of said second plurality of ultra-violet light curtains lies substantially orthogonal to a direction of movement of the divest tray by the third conveyor.

Optionally, each of said first and second plurality of ultra-violet light curtains uses light sources emitting ultra-violet radiation in a wavelength range of 280-100 nm or photon energy range of 4.43-12.4 eV.

The present specification also discloses a screening system for inspecting an object and sterilizing the object, comprising: a scanner having a housing defining an inspection volume, a radiation source coupled to the inspection volume, and a detector array coupled to the inspection volume, wherein the housing comprises an upstream side configured to allow the object to enter the inspection volume and a downstream side configured to allow the object to exit the inspection volume; an entry shroud having a first end and a second end, wherein the first end is coupled to the upstream side and wherein the entry shroud comprises a first frame extending between the first end and the second end; an exit shroud having a third end and a fourth end, wherein the third end is coupled to the downstream side and wherein the exit shroud comprises a second frame extending between the third end and the fourth end; a first plurality of ultraviolet light curtains coupled to the entry shroud, wherein the first plurality of ultraviolet light curtains are positioned between the first end and the second end; and a first conveyor extending at least from the second end to the first end of the entry shroud and a second conveyor extending at least from the third end to the fourth end of the exit shroud, wherein the first conveyor is configured to transport the object from the second end through the first plurality of ultra-violet light curtains to the inspection volume and the second conveyor is configured to transport the object from the inspection volume after scanning to the fourth end.

Optionally, each of the first conveyor and the second conveyor includes at least two belts and wherein the at least two belts having at least one split space between them.

Optionally, each of the first plurality of ultra-violet light curtains comprises a first ultra-violet light source positioned along a first side of the first frame, a second ultra-violet light source positioned along a second side of the first frame, wherein the second side opposes the first side, a third ultra-violet light source positioned along a third side of the first frame and a fourth plurality ultra-violet light source positioned along a fourth side of the first frame, wherein the third side opposes the fourth side. Optionally, the first ultra-violet light source, the second ultra-violet light source, and third ultra-violet light sources are positioned above belt surfaces of the first conveyor and are configured to illuminate a first surface, a second surface and a third surface, respectively, of the object while being transported by the first conveyor. Optionally, the fourth ultra-violet light source is positioned below the belt surfaces to direct ultra-violet light through a space in the belt surfaces to thereby illuminate a fourth surface of the object.

Optionally, each of the first plurality of ultra-violet light curtains comprises a first plurality of ultra-violet lights positioned along a first side of the first frame, a second plurality of ultra-violet lights positioned along a second side of the first frame, wherein the second side opposes the first side, a third plurality of ultra-violet lights positioned along a third side of the first frame and a fourth plurality of ultra-violet lights positioned along a fourth side of the first frame, wherein the third side opposes the fourth side. Optionally, the first plurality of ultra-violet lights, second plurality of ultra-violet lights and third plurality of ultra-violet lights are positioned above belt surfaces of the first conveyor and are configured to illuminate a first surface, a second surface and a third surface, respectively, of the object while being transported by the first conveyor. Optionally, the fourth plurality of ultra-violet lights is positioned below the belt surfaces to direct ultra-violet light through a space in the belt surfaces to thereby illuminate a fourth surface of the object.

Optionally, the first frame lies in a plane that is substantially orthogonal to a direction of movement of the object by the first conveyor.

Optionally, each of the first plurality of ultra-violet light curtains uses light sources emitting ultra-violet radiation in a wavelength range of 280-100 nm or photon energy in a range of 4.43-12.4 eV.

Optionally, the scanning system further comprises a lead lined cover coupled to at least one of the first frame and the second frame.

Optionally, the entry shroud comprises a first portion extending from the second end, wherein the first portion is coupled to a second portion, wherein the second portion is coupled to a third portion, wherein the third portion is coupled to the first end, and wherein the first portion and third portion are positioned at a right angle to each other.

Optionally, the entry shroud comprises a first portion extending from the second end, wherein the first portion is coupled to a second portion, wherein the second portion is coupled to a third portion, wherein the third portion is coupled to the first end, wherein a longitudinal axis of the first portion and a longitudinal axis of the third portion are parallel to each other, and wherein the second portion is skewed at an angle of 45 degrees relative to the longitudinal axes of both the first portion and the third portion.

Optionally, the entry shroud comprises a first portion extending from the second end, wherein the first portion is coupled to a second portion, wherein the second portion is coupled to a third portion, wherein the third portion is coupled to the first end, and wherein the first portion and third portion are configured such that a longitudinal axis of the first portion and a longitudinal axis of the third portion are neither parallel nor orthogonal to each other.

Optionally, the object is a parcel or a baggage.

The aforementioned and other embodiments of the present shall be described in greater depth in the drawings and detailed description provided below.

### BRIEF DESCRIPTION OF THE DRAWINGS

These and other features and advantages of the present specification will be further appreciated, as they become better understood by reference to the following detailed description when considered in connection with the accompanying drawings:

FIG. 1A is a perspective view of a curtained shroud coupled to a screening system, in accordance with some embodiments of the present specification;

FIG. 1B is a schematic diagram illustrating a first top view of the shroud of FIG. 1A, in accordance with some embodiments of the present specification;

FIG. 1C is a schematic diagram illustrating a second top view of the shroud of FIG. 1A, in accordance with some embodiments of the present specification;

FIG. 1D is a schematic diagram illustrating a third top view of the shroud of FIG. 1A, in accordance with some embodiments of the present specification;

FIG. 2A shows a perspective view of a curtain-less shroud coupled to a screening system, in accordance with some embodiments of the present specification;

FIG. 2B is a schematic diagram illustrating a first top view of the curtain-less shroud of FIG. 2A, in accordance with some embodiments of the present specification;

FIG. 2C is a schematic diagram illustrating a second top view of the curtain-less shroud of FIG. 2A, in accordance with some embodiments of the present specification;

FIG. 2D is a schematic diagram illustrating a third top view of the curtain-less shroud of FIG. 2A, in accordance with some embodiments of the present specification;

FIG. 2E shows first and second perspective views along with a plan view of a curtain-less shroud, in accordance with some embodiments of the present specification;

FIG. 3A shows a top view of a checkpoint security system deploying a tray return system that is configured to sterilize divest trays, in accordance with some embodiments of the present specification;

FIG. 3B shows divest stations, regions or areas with a tray being loaded onto an input conveyor lane, in accordance with some embodiments of the present specification;

FIG. 3C shows the tray being conveyed to a screening system of the checkpoint security system of FIG. 3A, in accordance with some embodiments of the present specification;

FIG. 3D shows the tray being diverted to a clear conveyor lane, in accordance with some embodiments of the present specification;

FIG. 3E shows reclaim stations, regions or areas with the tray being loaded onto a vertical elevator, in accordance with some embodiments of the present specification;

FIG. 3F shows the tray being loaded onto a return conveyor lane, in accordance with some embodiments of the present specification;

FIG. 3G shows the tray being transported onto the return conveyor lane, in accordance with some embodiments of the present specification;

FIG. 3H shows the tray being transported to the divest stations, regions or areas, in accordance with some embodiments of the present specification;

FIG. 3I shows a side elevation view of a portion of the clear conveyor lane and the return conveyor lane, in accordance with some embodiments of the present specification;

FIG. 3J shows a perspective view of the portion of the clear conveyor lane and the return conveyor lane, in accordance with some embodiments of the present specification;

FIG. 4 is a longitudinal schematic view of a real time tomography security scanning system having a circular locus of source points, in accordance with some embodiments;

FIG. 5 shows a perspective view of an X-ray security inspection machine, in accordance with some embodiments, in which the machine is deployed for use;

FIG. 6 is a flowchart of a plurality of exemplary steps of a method of sterilizing a parcel/baggage, in accordance with some embodiments of the present specification;

FIG. 7 is a flowchart of a plurality of exemplary steps of a first method of sterilizing a divest tray, in accordance with some embodiments of the present specification;

FIG. 8 is a flowchart of a plurality of exemplary steps of a second method of sterilizing a divest tray, in accordance with some embodiments of the present specification; and

FIG. 9 is a flowchart of a plurality of exemplary steps of a third method of sterilizing a divest tray, in accordance with some embodiments of the present specification.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated. It should be noted herein that any feature or component described in association with a specific embodiment may be used and implemented with any other embodiment unless clearly indicated otherwise.

As used herein, the indefinite articles “a” and “an” mean “at least one” or “one or more” unless the context clearly dictates otherwise.

An ‘entry shroud’ is a draping of material placed on an upstream side of a screening system where the bags/parcels being inspected enter the screening system. An ‘exit shroud’ is the a draping of material on the downstream side of the screening system where the bags/parcels exit the screening system. In this disclosure references to ‘shroud’ apply to both the entry and exit shrouds. A ‘profile plate’ refers to a device that replicates the tunnel entrance of a screening system in such a way that if a bag/parcel is either shaped or placed in a manner that prevents its entry into the screening system, the profile plate emits a signal indicating the same to an operator. Each shroud comprises a scanner end coupled with the screening system and a BHS/MHS end allowing bags/parcels being scanned to enter or exit the screening system. In various embodiments, the shroud is equipped to transmit or receive data signals comprising binary data.

As used herein, the term UV refers to ultraviolet (UV) light, which falls in the range of the EM spectrum between visible light and X-rays. It has frequencies of about 8×10″ to 3×10¹⁶ cycles per second, or hertz (Hz), and wavelengths of about 380 nanometers (1.5×10⁻⁵ inches) to about 10 nm (4×10⁻⁷ inches). UV light is generally divided into three sub-bands: UVA, or near UV (315-400 nm); UVB, or middle UV (280-315 nm); and UVC, or far UV (180-280 nm). The term UV, as used in this specification, may refer to any wavelength within this range. The terms UV light, UV-C light, UV light source, and UV-C light source may be used interchangeably throughout the specification and should be construed accordingly by those of ordinary skill in the art.

In embodiments, the present invention may include transceivers or transceiver modules each comprising a plurality of Receiver (Rx) elements and Transmitter (Tx) elements for communicating various forms of data. In various embodiments, a computing device may be employed to receive and process data signals and image data and may include an input/output controller, at least one communication interface and a system memory. The system memory includes at least one random access memory (RAM) and at least one read-only memory (ROM). These elements are in communication with a central processing unit (CPU) to enable operation of the computing device. In various embodiments, the computing device may be a conventional standalone computer or alternatively, the functions of the computing device may be distributed across a network of multiple computer systems and architectures. In some embodiments, execution of a plurality of sequences of programmatic instructions or code, which are stored in one or more non-volatile memories, enable or cause the CPU of the computing device to perform various functions and processes such as, for example, performing tomographic image reconstruction for display on a screen. In alternate embodiments, hard-wired circuitry may be used in place of, or in combination with, software instructions for implementation of the processes of systems and methods described in this application. Thus, the systems and methods described are not limited to any specific combination of hardware and software.

### Parcel/Baggage Sterilization System

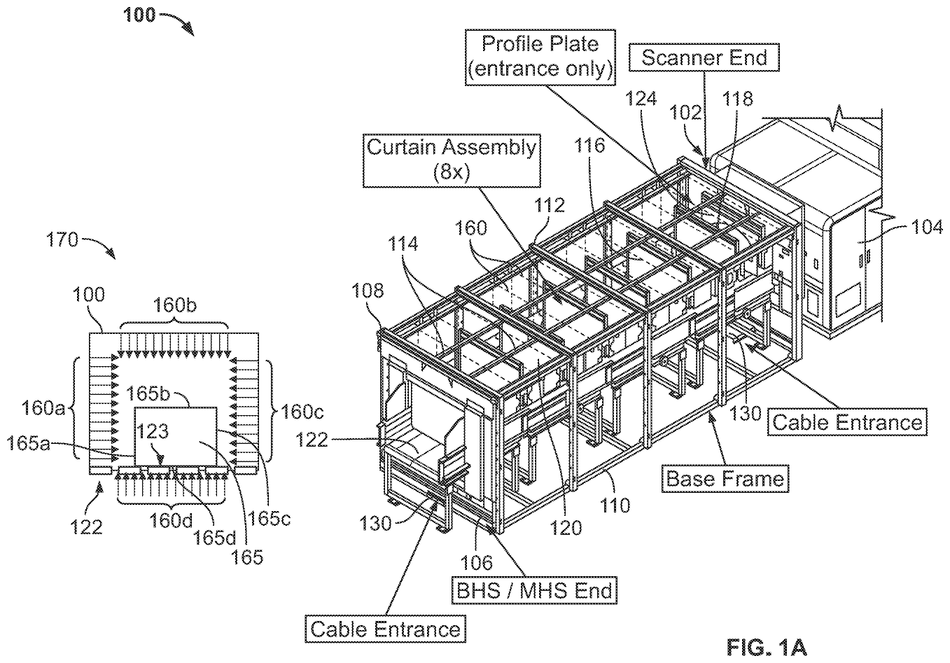

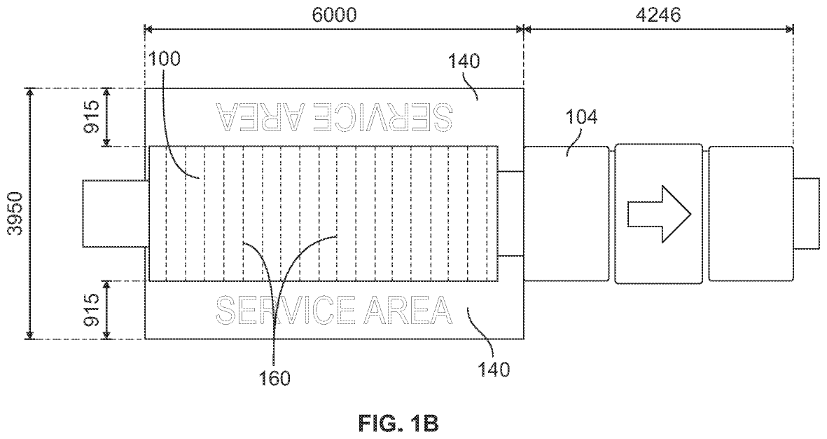

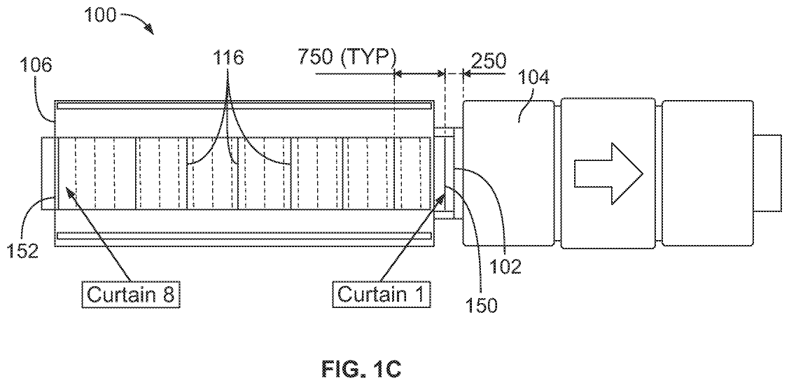

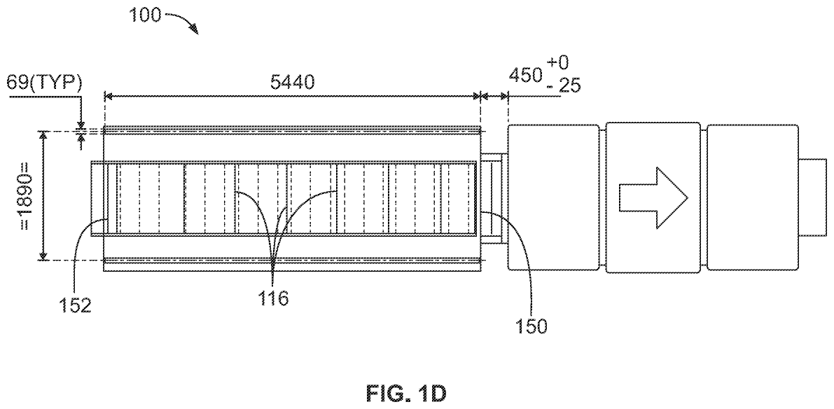

FIG. 1A shows a perspective view of a curtained shroud 100 coupled to a screening system 104, in accordance with some embodiments of the present specification. The curtained shroud 100 comprises a scanner end 102 coupled with the screening system 104 and a BHS/MHS (Baggage Handling System/Material Handling System) end 106 allowing baggage and/or parcels (or packages) to be scanned to enter or exit the screening system 104.

In some embodiments, the shroud 100 has a transverse cross-section having a substantially rectangular shape that is defined and supported by a metal frame structure 108 comprising a base frame 110 and a top frame 112. In other embodiments, the shroud 100 may have a transverse cross-section having a substantially circular shape or any substantially quadrilateral shape such as, for example, substantially square or trapezoidal. The metal frame structure 108 supports a steel cover (not shown) preferably coated with a durable, long-lasting paint. Metal rails 114 coupled to the top frame 112 extend from the scanner end 102 to the BHS/MHS end 106 and support a plurality of curtain assemblies 116. A conveyor 122 extends at least from the BHS/MHS end 106 to the scanner end 102. In some embodiments, one or more conveyor belts 123 are mounted on the conveyor 122, within the shroud 100, in a manner such that the conveyor belts 123 are essentially flat, lay in a substantially horizontal plane and in alignment with each other. It should be appreciated that each of the entry and exit shrouds includes a conveyor (similar to the conveyor 122) so that a first conveyor of the entry shroud transports packages to the screening system 104 for scanning and a second conveyor of the exit shroud transports a scanned package from the screening system 104 after scanning.

Each of the plurality of curtain assemblies 116 comprises a frame 118 coupled to the metal rails 114 such that a plane of the frame 118 is perpendicular with respect to the metal rails 114, the one or more conveyor belts 123 and a direction of motion of a parcel or baggage on the one or more conveyor belts. A curtain 120 comprising narrow strips of material (for example, electrical grade phenolic sheet material having a linen fabric reinforcement) extends downwards from the frame 118 and has a first end coupled with the frame 118 and a second end terminating proximate to the one or more belts 123 of the conveyor 122. In embodiments, the curtains 120 of the curtain assemblies 116 reduce a risk of slippage of the parcel/baggage slippage 165 during conveyance. In embodiments, a plane of the curtain 120 is substantially perpendicular to the one or more conveyor belts 123 and a direction of motion of a parcel on the one or more conveyor belts. In embodiments a height of the conveyor 122 from the floor on which the shroud 100 is placed ranges from 863 mm to 913 mm. In embodiments, at least one curtain assembly 116 is positioned at the BHS/MHS end 106. In some embodiments, each entry and exit shroud 100 includes 8 curtain assemblies 116. In some embodiments, a distance between two consecutive curtain assemblies 116 is at least 750 mm. In various embodiments, the scanner end 102 comprises a profile plate 124 positioned proximate to or at an entrance of a scanning tunnel of the screening system 104.

The profile plate 124 is configured to emit a data signal to an operator if a bag/parcel has a shape, size or orientation that prevents its entry into the scanning tunnel of the screening system 104. In embodiments, the profile plate 124 is a device that replicates the scanning tunnel entrance in such a way that a bag/parcel that is out of place (that is, a bag/parcel that would not enter the scanning tunnel clearly or without obstruction) will cause the plate to emit a data signal. In various embodiments, the profile plate 124 may be an actual plate or a virtual plate in the form of a laser or ultrasonic scanner. Cable entrances 130, each having a width and height of approximately 220 mm and 95 mm respectively, are provided at floor level at the scanner and BHS/MHS ends 102, 106 of the shroud. In embodiments, BHS/MHS cables are routed through these cable entrances 130. In an embodiment, each entry and exit shroud 100 weighs approximately 2000 kg, and the weight is distributed evenly (on a flat floor) by the base frame 110. In some embodiments, weight of each entry and exit shroud 100 does not exceed 3245 kg.

In accordance with some aspects of the present specification, UV (ultraviolet) light sources are provided for disinfection and sterilization of baggage and parcels. In embodiments, a plurality of UV (Ultra Violet) light curtains 160 are positioned between the BHS/MHS end 106 and the scanner end 102 within the shroud 100. In some embodiments, the plurality of UV light curtains 160 is positioned within the entry shroud. In some embodiments, the plurality of UV light curtains 160 is positioned within the exit shroud. In some embodiments, the plurality of UV light curtains 160 is positioned within both the entry and exit shrouds.

In some embodiments, a density of presence of the UV light curtains 160 is at least Z UV light curtains/centimeter within the entry and/or exit shroud. In some embodiments, at least 1 UV light curtains 160 are positioned between the BHS/MHS end 106 and the scanner end 102 within the shroud 100. In some embodiments, at least 15 UV light curtains 160 are positioned between the BHS/MHS end 106 and the scanner end 102 within the shroud 100. In some embodiments, the number of UV light curtains 160 ranges from 1 to 50. In some embodiments, the number of UV light curtains 160 ranges from 15 to 21. In some embodiments, the number of UV light curtains 160 is 18. In some embodiments, at least two UV light curtains 160 are positioned within each space between the plurality of adjacent curtain assemblies 116. In some embodiments, consecutive UV light curtains 160 are spaced apart from each other by a distance in a range of 259 mm to 363 mm. In embodiments, each UV light curtain comprises at least one UV light source. In embodiments, each UV light curtain comprises four UV lights or light sources, wherein one UV light source is positioned on each of four sides of the UV light curtain. In embodiments, each light curtain comprises four UV-C light sources, one for each side. In embodiments, each UV-C light source is in the form of a UV-C tube. In embodiments, a UV-C tube may be comprised of a plurality of UV-C lights or UV-C light sources. In embodiments, each UV light curtain comprises a plurality of UV lights or UV light sources.

In some embodiments, conveyors positioned within both entry and exit shrouds 100 are configured to operate at a speed of 0.5 meters per second. Accordingly, in some embodiments, the plurality of UV light curtains 160 provide a total UV exposure time ranging from 5 to 25 seconds, within the curtained shroud 100 (which is configured as a ‘straight’ shroud without bends), considering both entry and exit shroud lengths. In some other embodiments, the plurality of UV light curtains 160 provide a total UV exposure time ranging from 19 to 22 seconds, within the curtained shroud 100 (which is configured as a ‘straight’ shroud without bends), considering both entry and exit shroud lengths. In some other embodiments, the plurality of UV light curtains 160 provide a total UV exposure time of about 21.76 seconds, within the curtained shroud 100 (which is configured as a ‘straight’ shroud without bends), considering both entry and exit shroud lengths.

View 170 illustrates a substantially rectangular cross-section of the shroud 100, in accordance with some embodiments. The cross-sectional view 170 shows a substantially cuboid shaped parcel 165 (or baggage) positioned on one or more conveyor belts 123 and being transported (or conveyed) through the shroud 100 using the conveyor 122. In some embodiments, a UV light curtain 160 comprises at least one first UV light source 160a positioned along a first side within the shroud 100, at least one second UV light source 160c positioned along a second side (opposing the first side) within the shroud 100 and at least one third UV light source 160b positioned along a third side (or a top side) within the shroud 100. The at least one first, at least one second, and at least one third UV light sources 160a, 160c, 160b are positioned above the surfaces of the one or more conveyor belts 123 and respectively illuminate first, second and third sides/surfaces 165a, 165c, 165b of the parcel 165 as it is moved by the conveyor 122.

In some embodiments, at least the third UV light source(s) 160b in at least a portion of the UV light curtains 160 are oriented such that they emit UV light beams at an acute angle relative to a longitudinal axis of the shroud 100 in a horizontal plane and in a direction of conveyance of the parcel 165 to illuminate a trailing surface of the parcel 165. The at least third UV light source(s) 160b in at least a portion of the UV light curtains 160 are oriented such that they emit UV light beams at an acute angle to the longitudinal axis of the shroud 100 (in a horizontal plane) and in a direction opposite to the conveyance direction of the parcel 165 to illuminate a leading surface of the parcel 165. In some embodiments, light beams emanating from consecutive light curtains 160 may overlap to achieve sufficient coverage and exposure of the parcel 165.

In some embodiments, surfaces of the one or more conveyor belts 123 are layered with a germicidal coating to disinfect a bottom or fourth side/surface 165d that rests on the one or more conveyor belts 123. Alternatively or additionally, in some embodiments, the at least one fourth UV light source 160d is positioned along a fourth side within the shroud 100 and below the surfaces of the one or more conveyor belts 123 so as to beam UV light through splits spaces between the one or more conveyor belts 123 thereby illuminating the bottom or fourth side/surface 165d of the parcel 165. In some embodiments, a plane of a UV light curtain 160, comprising a mesh of light beams emanating from the at least one first, at least one second, at least one third and optionally at least one fourth UV light sources 160a, 160b, 160c and 160d, lies substantially orthogonal to the horizontal one or more conveyor belts 123 as well as to a direction of movement of the parcel 165 by the conveyor 122.

In some embodiments, each of the plurality of UV light curtains 160 uses UV-C light sources such as, for example, Mercury Vapor Lamps or LEDs (Light Emitting Diodes) emitting UV light/radiation in a wavelength range of 100 to 280 nm (shortwave UV-C) or photon energy range of 4.43-12.4 eV or 0.710-1.98 aJ. Such UV-C radiation is germicidal and effective in killing 99.9% of germs (bacteria and viruses) thereby sterilizing each parcel or baggage that is conveyed through the shroud 100. In embodiments, UV-C exposure (in a wavelength range of 222 to 254 nm) for a duration ranging from 5 to 10 seconds is effective in deactivating 99.9% of viruses. It should be appreciated that the enclosed and leaded configuration of the shroud 100 (entry and exit) encompasses a non-intrusive volume where the purpose of sterilization of the parcel 165, using the plurality of UV light curtains 160, can be performed with safety to all personnel working in the vicinity.

FIG. 1B is a schematic diagram illustrating a first top view of the shroud 100 shown in FIG. 1A, in accordance with some embodiments of the present specification. As shown in the figure, a service area 140 having a length of approximately 6000 mm and a width of approximately 915 mm surrounds the shroud 100. A height of the service area 140 from the floor is approximately 2300 mm. The service area 140 is required to be kept free from any obstruction during operation of the screening system 104 coupled with the shroud 100. In an embodiment the length of the screening system 104 is approximately 4246 mm and a width of the shroud 100 and the service area is approximately 3950 mm, as shown in FIG. 1B. Also shown is the plurality of UV light curtains 160.

FIGS. 1C and 1D are schematic diagrams illustrating second and third top views of the shroud 100 showing spacing between the plurality of curtain assemblies 116, in accordance with some embodiments of the present specification. Referring to FIGS. 1C and 1D, in some embodiments, the plurality of curtain assemblies 116 are spaced at least 750 mm apart. In embodiments, positions of the curtain assemblies 116 can be adjusted to avoid interfering with the conveyor PEC (Pulsed Eddy Current) sensors. In various embodiments a vertical length of the curtain 120 (FIG. 1A) is approximately 920 mm. Also shown is the plurality of UV light curtains 160.

In an embodiment, eight curtains are positioned within the shroud 100 (that is, within entry or exit shroud), where a first curtain 150 is proximate the scanner end 102 and a last curtain 152 is proximate the BHS/MHS end 106. The conveyor beds and their PEC sensors positions is designed to allow for the correct spacing of the curtains so as not to hinder the baggage throughput through the shroud 100. In an embodiment, the space between the curtain 150 and the scanner end 102 is adjustable, however, in some embodiments, a minimum distance between the curtain 150 and the screening system 104 is 250 mm. The curtain 152 is positioned as close to the BHS/MHS end 106 as possible. In an embodiment, the distance between the first curtain 150 and the last curtain 152 is approximately 5440 mm. In an embodiment, an internal width of the shroud 100 is 1890 mm. In an embodiment, a point loading of the shroud 100 is 0.27 kg/cm2.

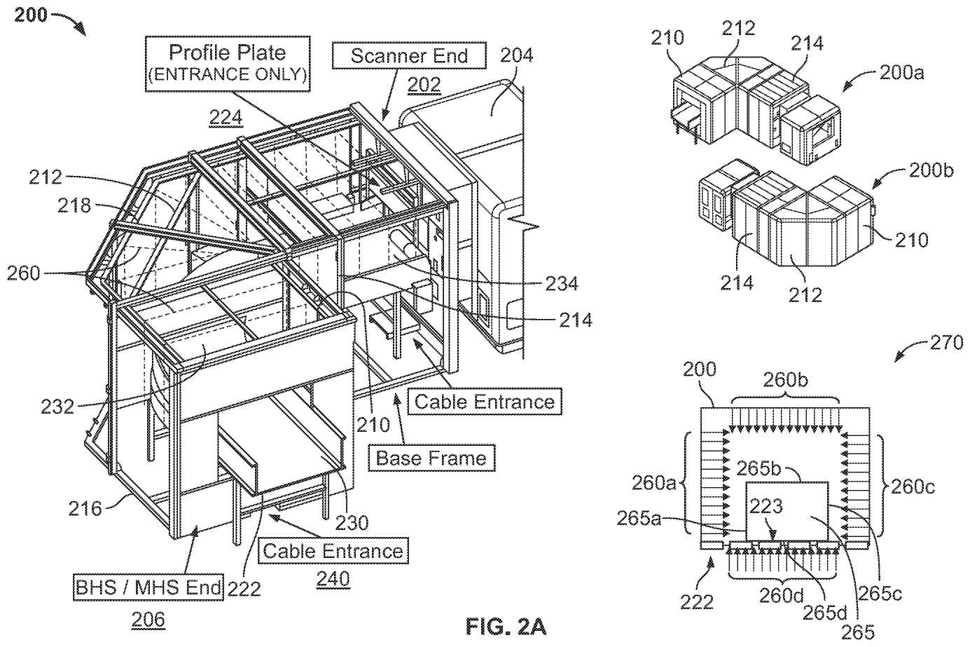

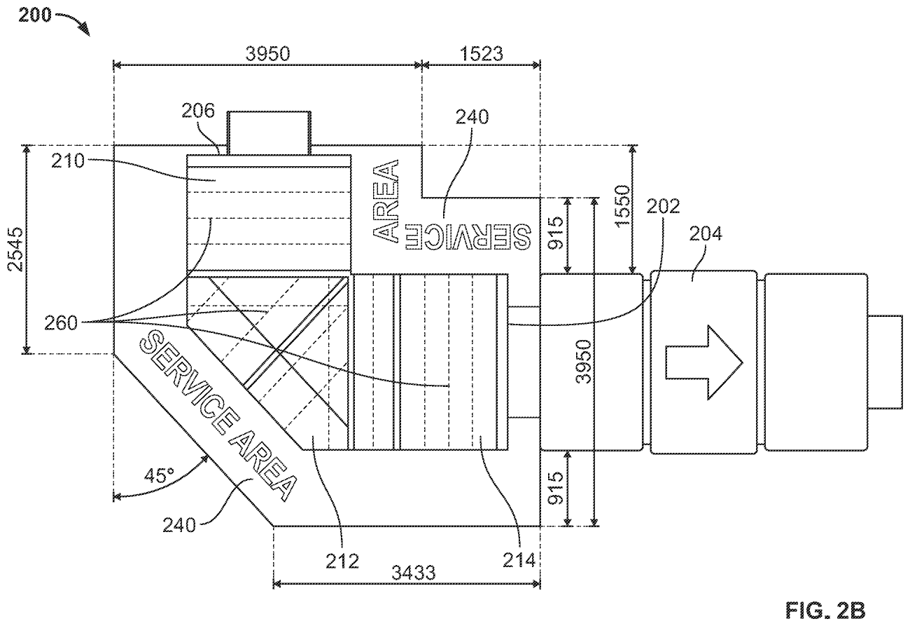

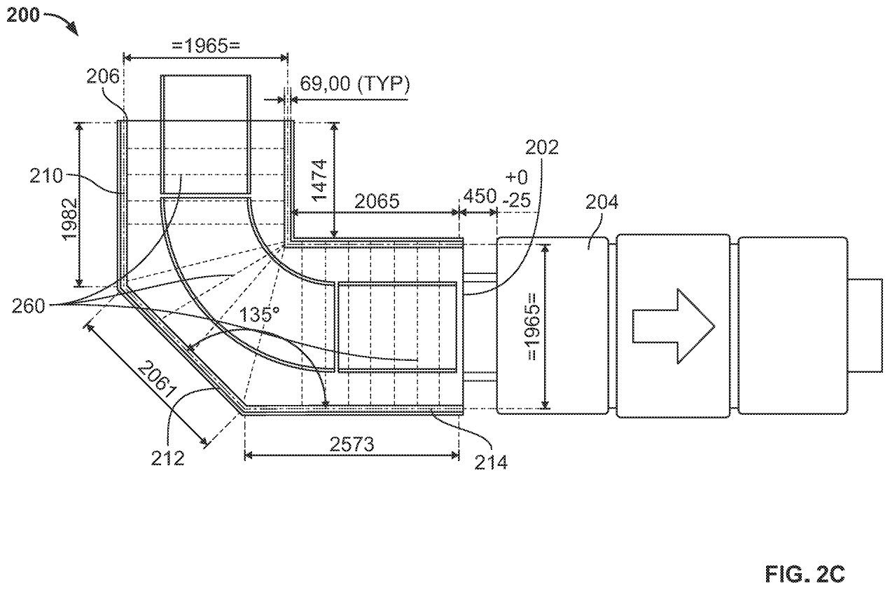

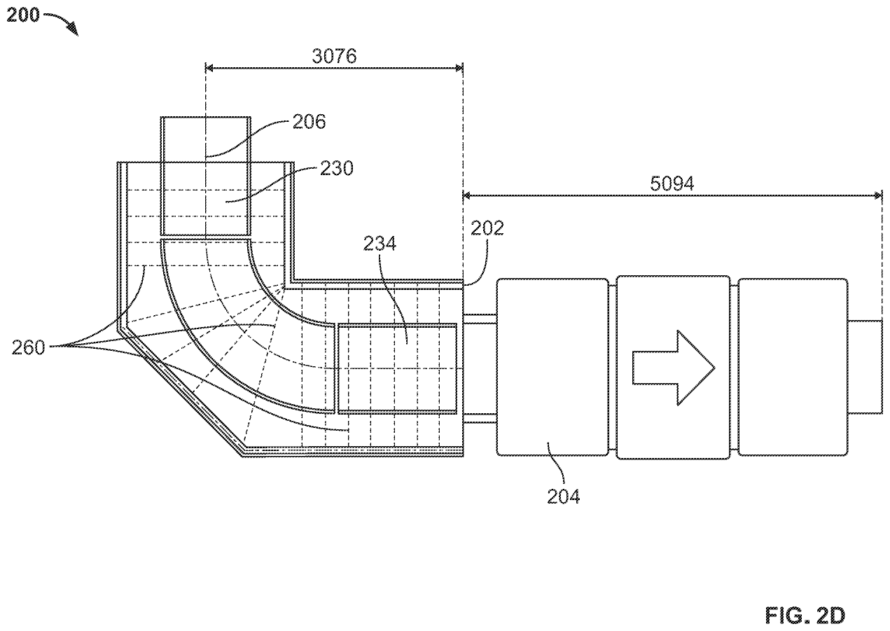

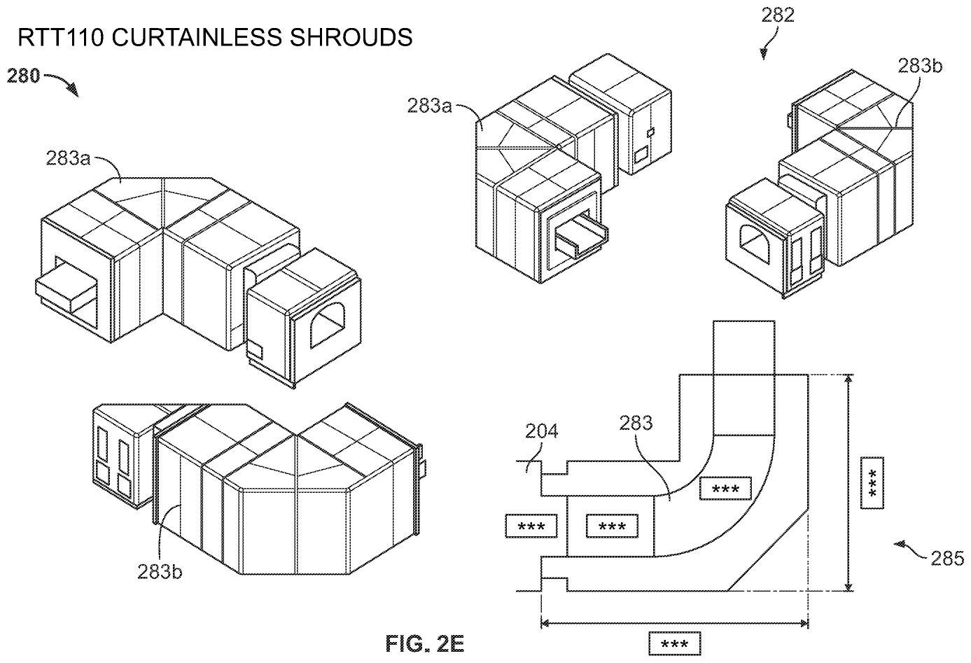

FIG. 2A shows a perspective view of a curtain-less shroud 200 coupled to a screening system 204, in accordance with some embodiments of the present specification. The shroud 200 comprises a scanner end 202 coupled with the screening system 204 and a BHS/MHS end 206 allowing parcels and/or baggage being scanned to enter or exit the screening system 204. In some embodiments, the shroud 200 comprises a first portion 210 extending from the BHS/MHS end 206 and coupled with a curved second portion 212 which in turn is coupled with a third portion 214 terminating in the scanner end 202. In some embodiments, the first and third portions 210, 214 respectively have a transverse cross-section of substantially rectangular shape. In other embodiments, the first and third portions 210, 214 may have a transverse cross-section of substantially circular or other quadrilateral shapes such as, for example, substantially square or trapezoidal. Also, shown in FIG. 2A are entry and exit shrouds 200a, 200b having first and third portions 210, 214 configured at a right angle to each other, in accordance with some embodiments. That is, the first portion 210 has a first longitudinal axis and the second portion 214 has a second longitudinal axis such that the first and second longitudinal axes are oriented at a right angle to each other. The perpendicular configuration of the shroud 200 provides additional advantage compared to the straight shroud 100 in terms of space saving and adaptability to different BHS designs. It should be noted that the use of lead curtain-less shrouds are, in some embodiments, advantageous as they are more efficient in allowing small and lighter weight parcels in passing through the conveyance. When using curtains, small and lightweight parcels may be dragged by the curtains, thereby impacting flow and throughput.

The shroud 200 is defined and supported by a metal frame structure 208 comprising a base frame 216 and a top frame 218. The metal frame structure 208 supports a steel cover (not shown) coating with a durable, long lasting paint. A conveyor 222 extends, within the shroud 200, at least from the BHS/MHS end 206 to the scanner end 202. In some embodiments, one or more conveyor belts 223 are mounted on the conveyor 222, within the shroud 200, such that the conveyor belts 223 are essentially flat lying in a horizontal plane and in alignment with each other.

In embodiments, a height of the conveyor 222 from the floor on which the shroud 200 is placed ranges from 863 mm to 913 mm. In an embodiment, as shown in FIG. 2A the conveyor 222 comprises a first straight portion 230 proximate the BHS/MHS end 206 wherein parcel/baggage to be inspected may be placed on said conveyor, a second straight portion 234 terminating in the scanner end 202 enabling the parcel/baggage to pass in to the scanner 204, and a curved portion 232 (not clearly visible in FIG. 2A) coupling the first and the second portions and enclosed by the curved second portion 212 of the shroud 200.

In various embodiments, the scanner end 202 comprises a profile plate 224 coupled with the screening system 204. The profile plate 224 emits a data signal if a bag/parcel is shaped, sized or placed in a manner that prevents its entry into the scanner 204, for indicating the same to an operator. A cable entrance 240 having a width and height of approximately 220 mm and 95 mm respectively is provided at floor level in the center of the panels at the scanner and BHS/MHS ends 202, 206 of the shroud 200. In embodiments, all BHS/MHS cables are routed through these cable entrances. In an embodiment, each entry and exit shroud 200 weighs approximately 2000 kg, and the weight is distributed evenly (on a flat floor) by the base frame 216. The point loading is 0.31 kg/cm2. In an embodiment, the curtainless-shroud 200 is designed to accommodate bags/parcels with a maximum height of 530 mm. In some embodiments, weight of each entry and exit shroud 200 does not exceed 3245 kg.

FIG. 2B illustrates a schematic diagram of the shroud 200 depicted in FIG. 2A, in accordance with an embodiment of the present specification. As shown in the figure, a service area 240 having a width of approximately 915 mm surrounds the shroud 200. A height of the service area 240 from the floor is approximately 2300 mm. The service area is required to be kept free from any obstruction during operation of the screening system 204 coupled with the shroud 200. In an embodiment the length of the screening system 204 is approximately 4246 m and a width of the shroud 200 plus the service area 240 is approximately 3950 m as shown in FIG. 2B. In an embodiment, lengths of the first and third portions 210, 214 are 2545 mm and 3433 mm, respectively. In some embodiments, the curved second portion 212 is skewed at an angle of 45 degrees with respect to a longitudinal direction of the first and third portions 210, 214 respectively. Also shown is a plurality of UV light curtains 260 positioned within the shroud 200.

FIG. 2C illustrates another schematic diagram of the shroud 200 depicted in FIG. 2A, in accordance with an embodiment of the present specification. As shown in the figure, in an embodiment, lengths of the first rectangular portion 210, the curved second portion 212 and the third rectangular portion 214 are 1982 mm, 2061 mm, and 2573 mm respectively. Also, in an embodiment the length of the screening system 204 is approximately 5094 mm. In some embodiments, the curved second portion 212 makes an obtuse angle of 135 degrees with respect to a longitudinal direction of the third portion 214. FIG. 2D illustrates another schematic diagram of the shroud 200 depicted in FIG. 2A, in accordance with an embodiment of the present specification. In an embodiment, as shown in FIG. 2D a width of the shroud 200 measured from the center of the conveyor bed 230 at the BHS/MHS end 206 to the scanner end 202 is approximately 3076 mm. Also shown is the plurality of UV light curtains 260 positioned within the shroud 200.

In some embodiments, at least 12 UV light curtains 260 are positioned between the BHS/MHS end 206 and the scanner end 202 within the shroud 200. In some embodiments, the number of UV light curtains 260 ranges from 1 to 25. In some embodiments, the number of UV light curtains 260 ranges from 12 to 14. In some embodiments, consecutive UV light curtains 260 are spaced apart from each other by a distance in a range of 363 mm to 423 mm.

In some embodiments, conveyors in both entry and exit shrouds 200 are configured to operate at a speed of 0.5 meters per second. Accordingly, in some embodiments, the plurality of UV light curtains 260 provide a total UV exposure time ranging from 5 to 25 seconds, within the angled curtain-less shroud 200, considering both entry and exit shroud lengths. In some other embodiments, the plurality of UV light curtains 260 provide a total UV exposure time ranging from 19 to 22 seconds, within the angled curtain-less shroud 200, considering both entry and exit shroud lengths. In yet some other embodiments, the plurality of UV light curtains 260 provide a total UV exposure time of about 20.3 seconds, within the angled curtain-less shroud 200, considering both entry and exit shroud lengths.

In embodiments, each UV light curtain comprises at least one UV light source. In embodiments, each UV light curtain comprises four UV light sources, wherein one UV light source is positioned on each of four sides of the UV light curtain. In embodiments, each light curtain comprises four UV-C light sources, one for each side. In embodiments, each UV-C light source is in the form of a UV-C tube. In embodiments, each UV light curtain comprises a plurality of UV light sources. In embodiments, a UV-C tube may be comprised of a plurality of UV-C light sources.

Referring back to FIG. 2A, view 270 illustrates a substantially rectangular cross-section of the shroud 200, in accordance with some embodiments. The cross-sectional view 270 shows a substantially cuboid shaped parcel 265 (or baggage) positioned on one or more conveyor belts 223 and being transported (or conveyed) through the shroud 200 using the conveyor 222. In some embodiments, a UV light curtain 260 comprises at least one first UV light source 260a positioned along a first side within the shroud 200, at least one second UV light source 260c positioned along a second side (opposing the first side) within the shroud 200 and at least one third UV light source 260b positioned along a third side (or a top side) within the shroud 200. The at least one first, at least one second, and at least one third UV light sources 260a, 260c, 260b are positioned above the surfaces of the one or more conveyor belts 223 and respectively illuminate first, second and third sides/surfaces 265a, 265c, 265b of the parcel 265 as it is moved by the conveyor 222.

In some embodiments, at least the third UV light source(s) 260b in at least a portion of the UV light curtains 260 are oriented such that they emit UV light beams at an acute angle relative to a longitudinal axis of the shroud 200 in a horizontal plane and in a direction of conveyance of the parcel 265 to illuminate a trailing surface of the parcel 265. The at least third UV light source(s) 260b in at least a portion of the UV light curtains 260 are oriented such that they emit UV light beams at an acute angle to the longitudinal axis of the shroud 200 (in a horizontal plane) and in a direction opposite to the conveyance direction of the parcel 265 to illuminate a leading surface of the parcel 265. In some embodiments, light beams emanating from consecutive light curtains 260 may overlap to achieve sufficient coverage and exposure of the parcel 265.

In some embodiments, each of the plurality of UV light curtains 260 uses UV-C light sources such as, for example, Mercury Vapor Lamps or LEDs (Light Emitting Diodes) emitting UV light/radiation in a wavelength range of 100 to 280 nm (shortwave UV-C) or photon energy range of 4.43-12.4 eV or 0.710-1.98 aJ. Such UV-C radiation is germicidal and effective in killing 99.9% of germs (bacteria and viruses) thereby sterilizing each parcel or baggage that is conveyed through the shroud 200. It should be appreciated that the enclosed and leaded configuration of the shroud 200 (entry and exit) encompasses a non-intrusive volume where the purpose of sterilization of the parcel 265, using the plurality of UV light curtains 260, can be performed with safety to all personnel working in the vicinity.

FIG. 2E shows a first perspective view 280 and a second perspective view 282 of entry and exit curtain-less shrouds 283a, 283b and 283c, 283d. Also shown is a plan view 285 of a line diagram of a curtain-less shroud 283 coupled to the screening system 204.

In an embodiment, an alignment conveyor is positioned prior to the entry shroud and one side wall is offset from the center line of conveyors that feed the screening system 104 by half a maximum bag/parcel width. This centralizes the maximum bag/parcel width with the scanning tunnel of the screening system 104. In another embodiment, a centralizing conveyor is used, which centralizes all bags/parcels.

In an embodiment the shrouds described in the present specification operate at a voltage level of approximately 220V+/−10% 50 Hz, while in another embodiment the operating voltage is approximately 110V+/−10% 60 Hz (single phase). In various embodiments, the shrouds run on approximately 10A current including in-rush. A mains lead which is approximately 15 m long is provided, and in an embodiment is hard wired to a junction box with a10A type C main circuit board.

In various embodiments a plurality of signals derived from the shrouds, are passed to a BHS/MHS by the scanner coupled with the shroud. In an embodiment, a ‘door open’ signal signifying that an access to the scanner is open is passed to the BHS/MHS system. In another embodiment, an ‘e-stop pressed’ signal signifying that an electronic stopped button is pressed is passed on to the BHS/MHS system. In an embodiment, an ‘entry profile plate active’ signal conveying an active state of the profile plate is passed on to the BHS/MHS system.

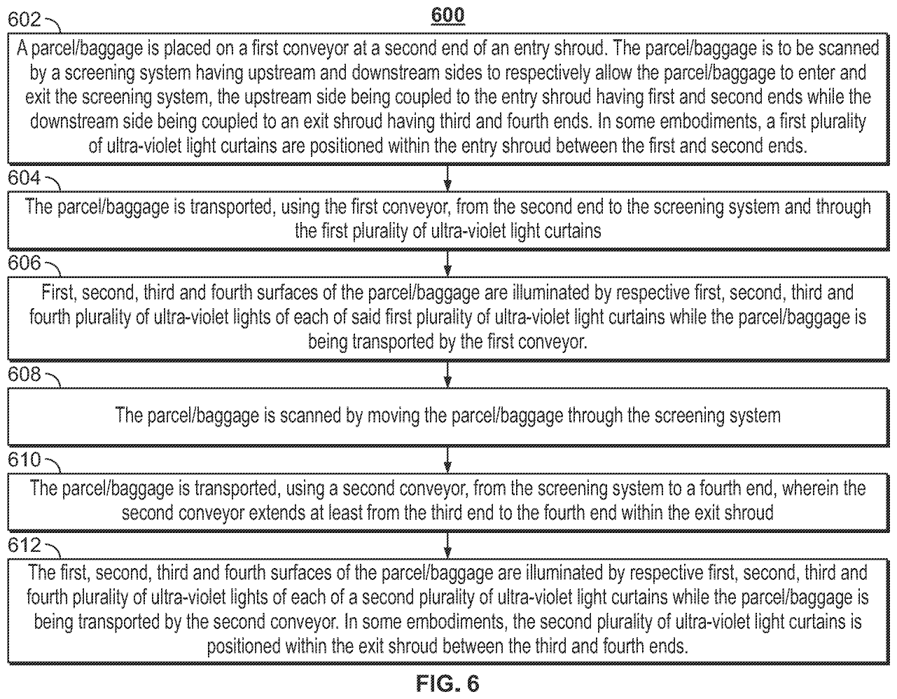

FIG. 6 is a flowchart of a plurality of exemplary steps of a method 600 of sterilizing a parcel/baggage, in accordance with some embodiments of the present specification. In various embodiments, the method 600 is implemented in any of the system 100 of FIGS. 1A, 1B, 1C and 1D or system 200 of FIGS. 2A, 2B, 2C and 2D. In embodiments, the method 600 is directed towards sterilizing the parcel/baggage to be scanned by a screening system having upstream and downstream sides to respectively allow the parcel/baggage to enter and exit the screening system, the upstream side being coupled to an entry shroud having first and second ends while the downstream side being coupled to an exit shroud having third and fourth ends, wherein a first plurality of ultra-violet light curtains are positioned within the entry shroud between the first and second ends.

At step 602, the parcel/baggage is placed on a first conveyor at the second end, wherein the first conveyor extends at least from the second end to the first end within the entry shroud. At step 604, the parcel/baggage is transported, using the first conveyor, from the second end to the screening system through the first plurality of ultra-violet light curtains, wherein each of the first plurality of ultra-violet light curtains has at least one ultra-violet light source positioned along a first side, at least one ultra-violet light source positioned along a second side opposing the first side, at least one ultra-violet light source positioned along a third side and at least one ultra-violet light source positioned along a fourth side opposing the third side.

At step 606, first, second, third and fourth surfaces of the parcel/baggage are illuminated by respective first, second, third and fourth ultra-violet light sources of each of said first plurality of ultra-violet light curtains while the parcel/baggage is being transported by the first conveyor. At step 608, the parcel/baggage is scanned by moving the parcel/baggage through the screening system.

At step 610, the parcel/baggage is transported, using a second conveyor, from the screening system to the fourth end, wherein the second conveyor extends at least from the third end to the fourth end within the exit shroud.

In some embodiments, a second plurality of ultra-violet light curtains is positioned within the exit shroud between the third and fourth ends. Each of the second plurality of ultra-violet light curtains has at least one ultra-violet light source positioned along a first side, at least one ultra-violet light source positioned along a second side opposing the first side, at least one ultra-violet light source positioned along a third side and at least one ultra-violet light source positioned along a fourth side opposing the third side. Consequently, in such embodiments, at step 612, the first, second, third and fourth surfaces of the parcel/baggage are illuminated by respective first, second, third and fourth ultra-violet light sources of each of the second plurality of ultra-violet light curtains while the parcel/baggage is being transported by the second conveyor.

It should be appreciated that in some embodiments, only the first plurality of ultra-violet light curtains is deployed as a result of which the step 612 is not executed. In some embodiments, only the second plurality of ultra-violet light curtains is deployed as a result of which the step 606 is not executed. In some embodiments, both first and second plurality of ultra-violet light curtains is deployed as a result of which both steps 606 and 612 are executed.

### A Divest Tray Sterilization System Integrated in a Tray Return System

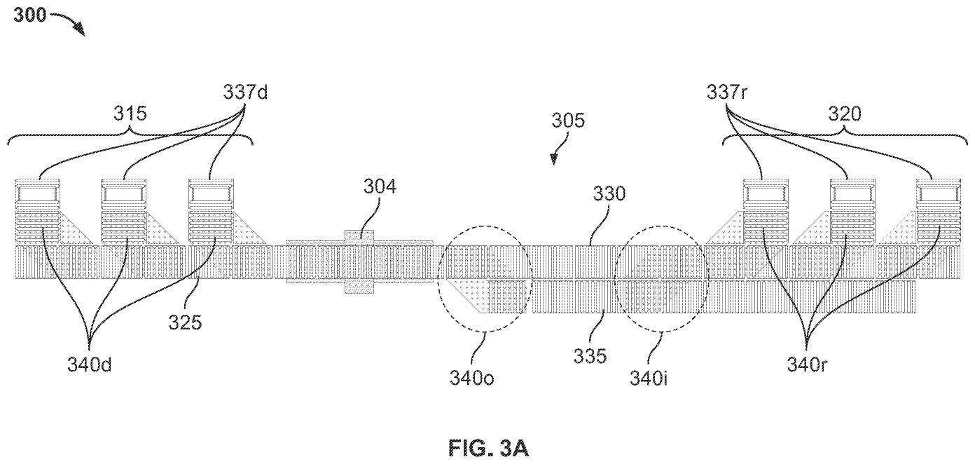

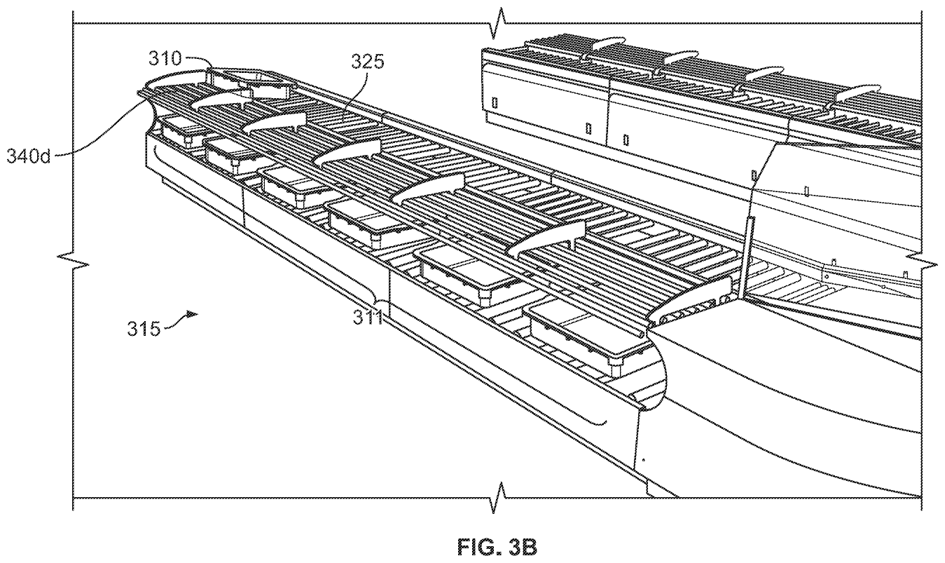

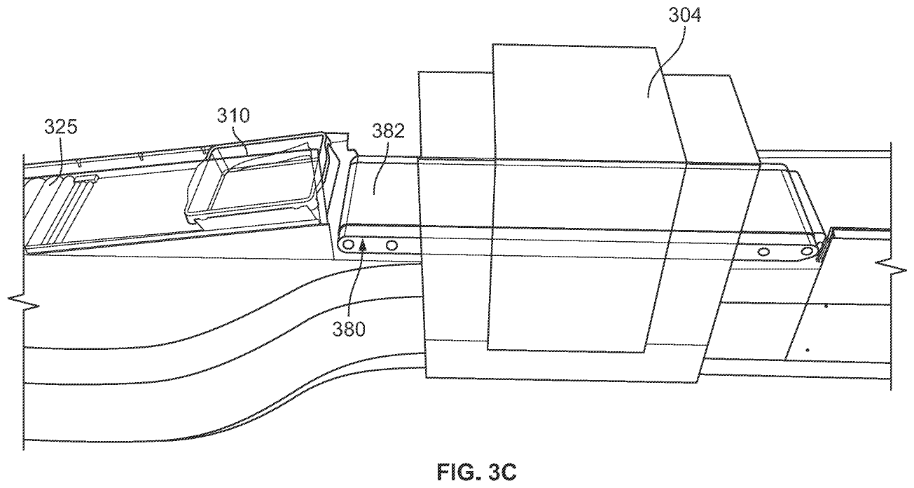

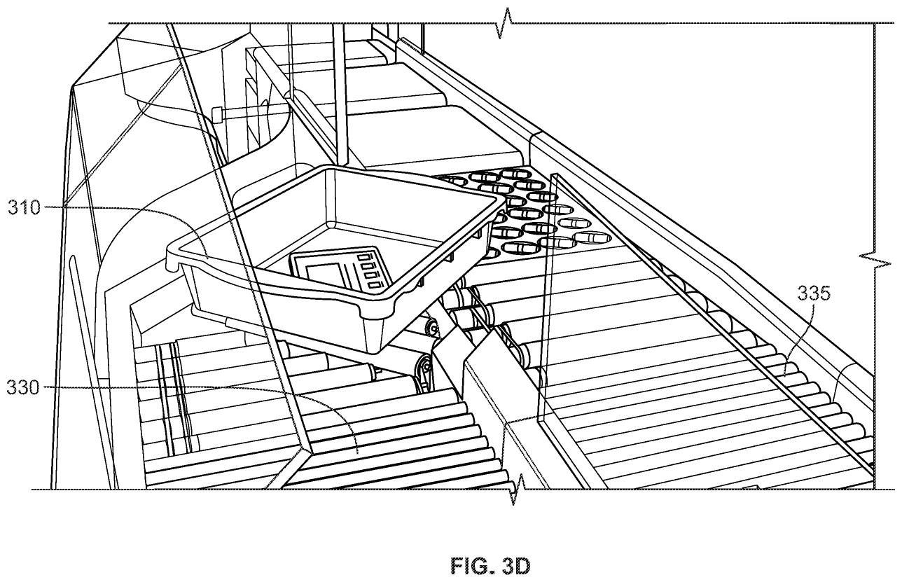

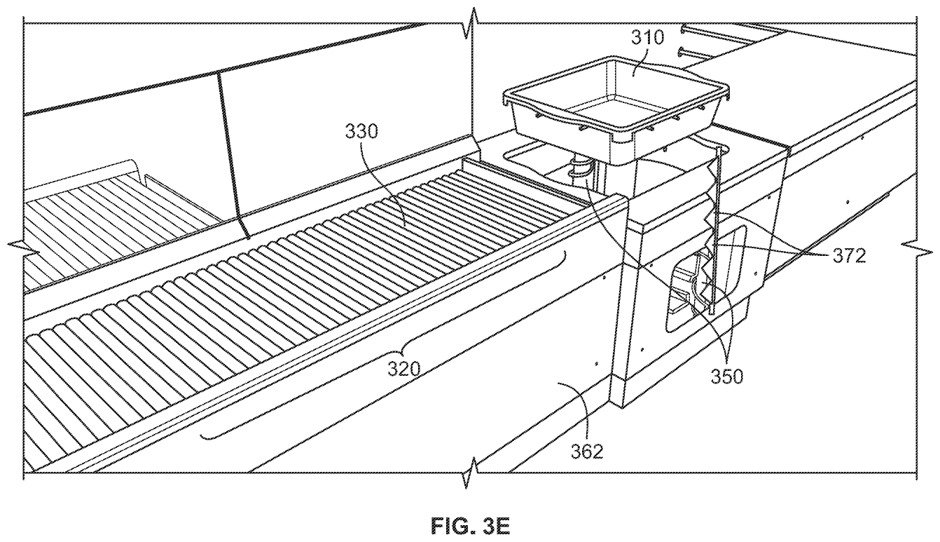

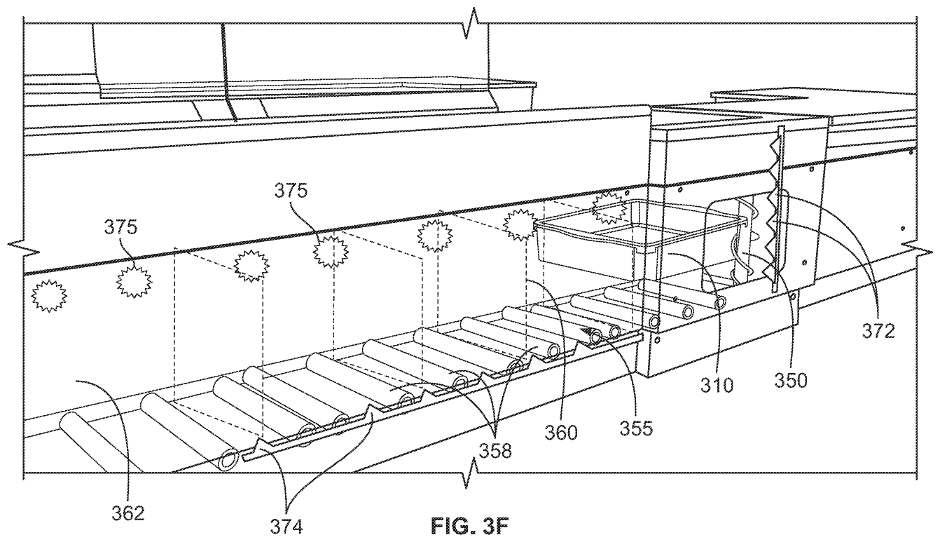

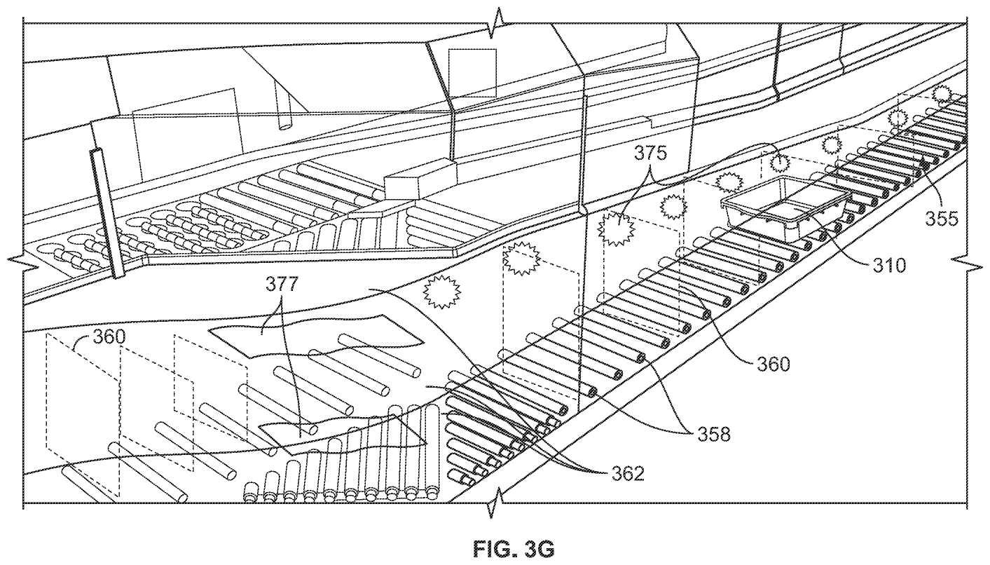

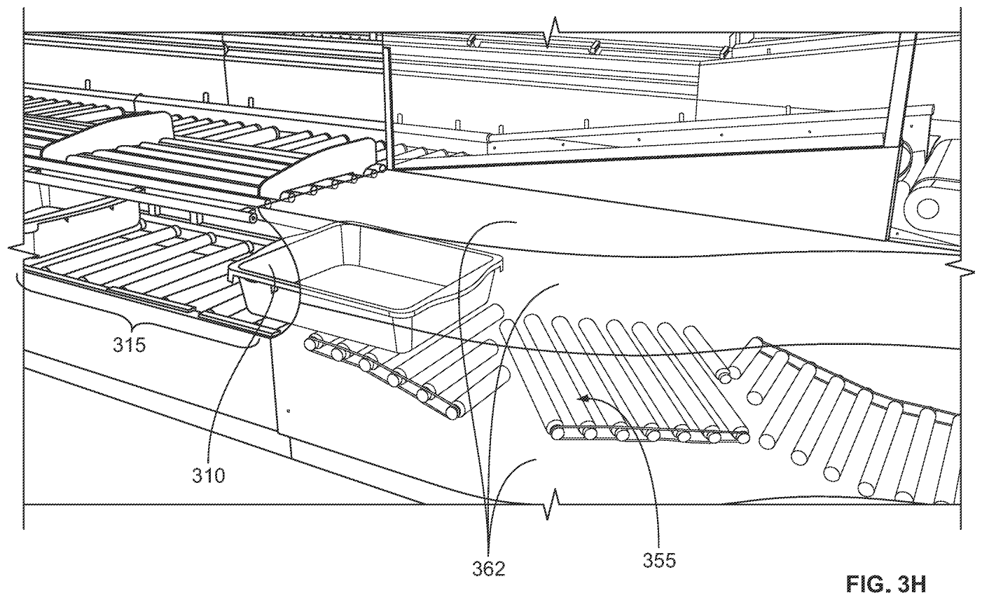

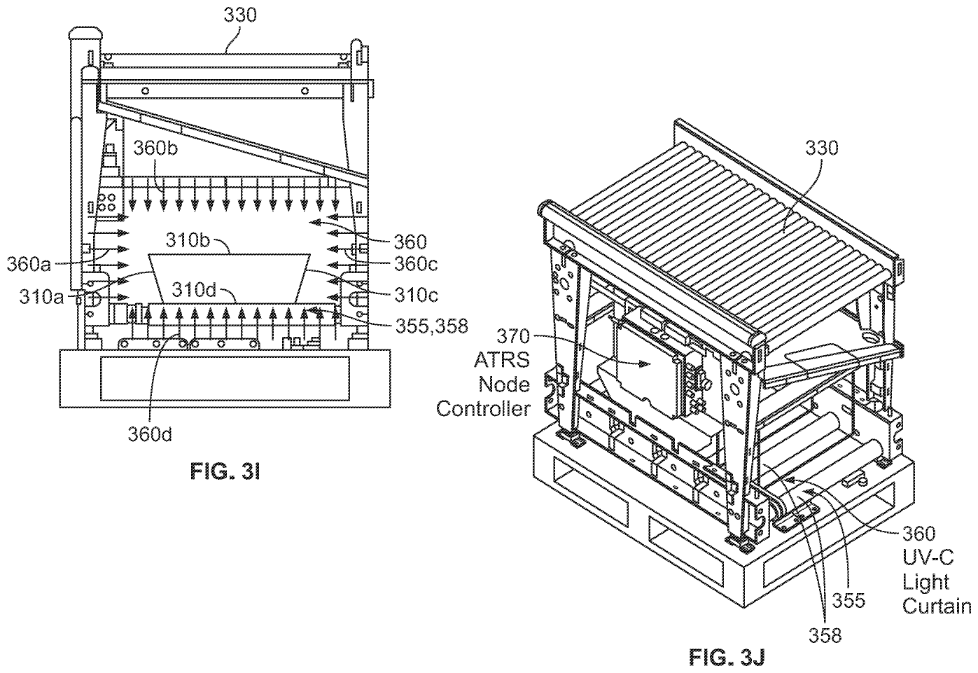

FIG. 3A shows a top view of a checkpoint security system 300 deploying a tray return system that is configured to sterilize divest trays, in accordance with some embodiments of the present specification. The system 300 comprises a screening system 304 and a tray return system 305. The tray return system 305 further comprises a plurality of divest stations 315, a plurality of reclaim stations 320, an input conveyor lane 325 positioned prior to the screening system 304 (or the scanning zone), output conveyor lanes comprising a clear conveyor lane 330 and a divert conveyor lane 335 positioned after the screening system 304 (or the scanning zone), a plurality of tray divert units 340 (340d, 340r, 340o, 340i) and a plurality of divest/reclaim queuing conveyor lanes 337 (337d, 337r) operating in the plurality of divest and reclaim stations 315, 320.

In embodiments of the present specification, and for illustrative purposes, the checkpoint security system 300 is considered to be an aviation checkpoint system while the screening system 304 is considered to be an X-ray baggage screening unit. It should be appreciated that this checkpoint system may be implemented in any location that requires restricting or controlling access by individuals, including, but not limited to, gates at bus stations, train stations, maritime ports, or any other form of public transportation, large public gathering spaces, such as stadiums, concert halls, or other venues, or government buildings.

FIGS. 3B through 3H show movement of a divest tray 310 along the tray return system 305 along with a system to sterilize the divest tray 310, in accordance with some embodiments of the present specification. Referring now to FIGS. 3A through 3H, during operation, once a passenger divests off personal and/or required items in the tray 310 and places the tray 310 onto a divest queuing conveyor lane 337d in one of the plurality of divest stations 315, a tray divert unit 340d enables the tray 310 to be diverted from the divest queuing conveyor lane 337d onto the input conveyor lane 325 to convey the tray 310 to the screening system 304. In embodiments, the screening system 304 is configured to generate its inspection result by the time that a leading edge of the tray 310 exits the screening system 304.

Specifically, FIG. 3B shows the tray 310 being diverted by the tray divert unit 340d onto the input conveyor lane 325 in the divest stations, regions or areas 315. A queue 311 of additional trays is also shown from where the passenger can pick the tray 310 to divest his items. FIG. 3C shows the tray 310 being conveyed on the input conveyor lane 325 to the screening system 304 for scanning.

As shown in FIG. 3A, if the scanned tray 310 is marked clear, the tray 310 proceeds along the clear conveyor lane 330 and towards the plurality of reclaim stations 320. A tray divert unit 340r enables the cleared tray 310 to be diverted from the clear conveyor lane 330 onto a reclaim queuing conveyor lane 337r in one of the plurality of reclaim stations 320. On the other hand, if the scanned tray 310 is marked as either ‘no result’ or threat, a tray divert unit 340o rejects or diverts the tray from the clear conveyor lane 330 onto the divert conveyor lane 335 that comprises a threat or ‘no result’ lane. In some embodiments, a tray divert unit 340i may divert a rejected tray that has subsequently been cleared from the divert conveyor lane 335 back onto the clear conveyor lane 330 for onward movement of the tray to one of the plurality of reclaim stations 320.

FIG. 3D shows the scanned tray 310 marked clear and being diverted (from the divert conveyor lane 335) to proceed along the clear conveyor lane 330. FIG. 3E shows a portion of the clear conveyor lane 330 conveying the tray 310 towards the reclaim stations, regions or areas 320. As the passenger reclaims his belongings, the empty tray 310 is conveyed into a tray elevator 350 that, in some embodiments, is positioned beyond the reclaim stations, regions or areas 320. FIG. 3F shows the tray elevator 350 bringing the tray 310 down and laying the tray 310 onto a return conveyor lane 355 that has a plurality of conveying rollers 358. In some embodiments, the return conveyor lane 355 is positioned at a second level or plane below a first level or plane of the clear conveyor lane 330. FIG. 3G shows the tray 310 being transported by the return conveyor lane 355 and towards the divest stations, regions or areas 315 (FIGS. 3B, 3H).

As can be seen in FIGS. 3F and 3G, the plurality of conveying rollers 358 have spaces or gaps in between. In accordance with some aspects of the present specification, a plurality of UV-C (Ultra Violet) light curtains 360 are positioned in the spaces or gaps between the conveying rollers 358. In some embodiments, a minimum of 8-10 light curtains, separated by at least one conveying roller is employed. In some embodiments, the plurality of UV light curtains 360 are positioned along a portion of the return conveyor lane 355 that lays between the tray elevator 350 and the screening system 304. In some embodiments, the plurality of UV light curtains 360 extend from the tray elevator 350 to an exit area of the screening system 304. In some embodiments, the plurality of UV light curtains 360 extend from the tray elevator 350 to an entry area of the screening system 304. The plurality of UV light curtains 360 ensure that the tray 310 has sufficient exposure time to the UV-C light source as it travels back to the front of the input conveyor lane 325 to ensure effective sterilization. In embodiments, the performance of the UV-C light source is a function of intensity and time. The higher the intensity, the less the time required to sterilize the trays. Depending on the sources used and the ozone producing capability, the exposure time may range from 2 seconds to 15 seconds, and more preferably, ranges from 5 seconds to 10 seconds. In embodiments, the conveyor speed would need to be adjusted to slow the return path of the tray during passage through the UV light curtains to ensure adequate exposure time. Once the tray passes through the curtains, the return speed can be increased slightly to help mitigate the delay on returning the tray back to the divestment area or module.

FIGS. 3I and 3J show side elevation and perspective views, respectively, of a portion of the clear conveyor lane 330 and the return conveyor lane 355, in accordance with some embodiments of the present specification. Referring now to FIGS. 3I and 3J, in some embodiments each of the plurality of UV light curtains 360 comprises at least one UV light source 360a positioned along a first side, at least one UV light source 360c positioned along a second side (opposing the first side), at least one UV light source 360b positioned along a third side (or a top side) and at least one UV light source 360d positioned along a fourth side (or a bottom side) opposing the third side. In embodiments, each light curtain comprises four UV-C light sources, one for each side. In embodiments, each UV-C light source is in the form of a UV-C tube.

In some embodiments, each of the plurality of UV light curtains 360 comprises a first plurality of UV light sources 360a positioned along a first side, a second plurality of UV light sources 360c positioned along a second side (opposing the first side), a third plurality of UV light sources 360b positioned along a third side (or a top side) and a fourth plurality of UV light sources 360d positioned along a fourth side (or a bottom side) opposing the third side.

The UV light sources 360a, 360b, 360c are positioned above the surfaces of the conveying rollers 358 and respectively illuminate first, third and second sides/surfaces 310a, 310b, 310c of the tray 310 as it is moved by the return conveyor lane 355. Also, placement of each of the plurality of UV light curtains 360 in the spaces or gaps between the conveying rollers 358 ensures that the fourth UV light source 360d positioned along the fourth side (or the bottom side) transmits UV light through the spaces or gaps between the conveying rollers 358 thereby illuminating the bottom side/surface 310d of the tray 310. The four-sided placement of the UV light sources, in each of the plurality of UV light curtains 360, ensures a 360-degree exposure of the tray 310 to the UV lights thereby achieving sterilization under the lips of the tray 310 as well as of surfaces 310a, 310b, 310c, and 310d.

In some embodiments, the at least one or plurality of UV light sources 360b in at least a first portion of the UV light curtains 360 are oriented such that they emit UV light beams at an acute angle to a conveyance direction of the tray 310 to illuminate a trailing surface of the tray 310. The at least one or plurality of UV light sources 360b in at least a second portion of the UV light curtains 360 are oriented such that they emit UV light beams at an acute angle to a direction opposite to the conveyance direction of the tray 310 to illuminate a leading surface of the tray 310. In some embodiments, light beams emanating from consecutive light curtains 360 may overlap to achieve sufficient coverage and exposure of the tray 310.

In some embodiments, each of the plurality of UV light curtains 360 uses UV-C light sources such as, for example, Mercury Vapor Lamps or LEDs (Light Emitting Diodes) emitting UV light/radiation in a wavelength range of 280-100 nm (shortwave UV-C) or photon energy range of 4.43-12.4 eV or 0.710-1.98 aJ. Such UV-C radiation is germicidal and effective in killing 99.9% of germs thereby sterilizing each tray that is conveyed using the return conveyor lane 355.

As shown in FIG. 3J, power for each of the plurality of UV light curtains 360 is obtained from a node control system 370 of the tray return system.

In some embodiments, a plane of a UV light curtain 360, comprising a mesh of light beams emanating from the at least one UV light 360a, at least one UV light 360b, at least one UV light 360c and at least one UV light 360d, lies substantially orthogonal to a plane of the return conveyor lane 355 as well as to a direction of movement of the tray 310 on the return conveyor lane 355.

Referring back to FIGS. 3F through 3H, as the tray 310 is transported, using the return conveyor lane 355, from the tray elevator 350 and towards the divest stations, regions or areas 315 (FIG. 3B, 3H), the tray 310 moves through the plurality of UV light curtains 360 that disinfect and sterilize the tray 310. FIG. 3H shows the tray 310 being moved (using the return conveyor lane 355) to the divest stations, regions or areas 315 after having being sterilized.

As can be observed from FIGS. 3E through 3H, the portion of the return conveyor lane 355 that includes the plurality of UV light curtains 360 is fully encompassed or enclosed within a housing 362 to prevent UV light exposure to personnel and passengers in the vicinity of the tray return system 305.

In some embodiments, a density of presence of the UV light curtains 360 is at least Z curtains/centimeter. In some embodiments, at least X number of UV light curtains 360 is positioned between the tray elevator 350 and the screening system 304 within the housing 362. In some embodiments, the total number of UV light curtains 360 ranges from Y to Z. In some embodiments, at least M number of UV light curtains 360 is positioned within each space between the conveying rollers 358. In some embodiments, consecutive UV light curtains 160 are spaced apart from each other by a distance of at least N and in a range of O to P. In some embodiments, a tray receives a total UV light exposure time of at least M minutes and in a range of N to O minutes.

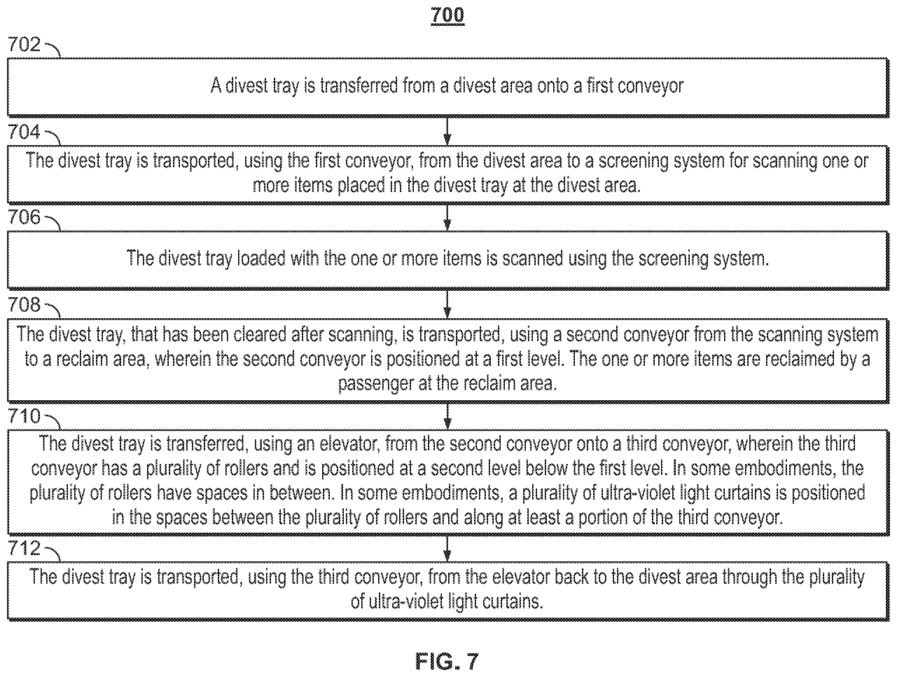

FIG. 7 is a flowchart of a plurality of exemplary steps of a method 700 of sterilizing a divest tray, in accordance with some embodiments of the present specification. In various embodiments, the method 700 is implemented in the checkpoint security system 300 of FIGS. 3A through 3J wherein the system 300 deploys a tray return system.

At step 702, the divest tray is transferred from a divest area onto a first conveyor. A passenger typically divests and places one or more items in the divest tray at the divest area. In some embodiments, the passenger is allowed to place the divest tray onto a divest queuing conveyor, at the divest area, and a tray divert unit enables the tray to be transferred from the divest queuing conveyor onto the first conveyor.

At step 704, the divest tray is transported, using the first conveyor, from the divest area to a screening system for scanning the one or more items placed in the divest tray at the divest area. Thereafter, at step 706, the divest tray loaded with the one or more items is scanned using the screening system.

At step 708, the divest tray, that has been cleared after scanning, is transported, using a second conveyor from the scanning system to a reclaim area, wherein the second conveyor is positioned at a first level. The one or more items are reclaimed by the passenger at the reclaim area. At step 710, the unloaded divest tray is transferred, using an elevator, from the second conveyor onto a third conveyor, wherein the third conveyor has a plurality of rollers and is positioned at a second level below the first level. In some embodiments, the plurality of rollers have spaces in between. In some embodiments, a plurality of ultra-violet light curtains is positioned in the spaces between the plurality of rollers and along at least a portion of the third conveyor.

At step 712, the divest tray is transported, using the third conveyor, from the elevator back to the divest area through the plurality of ultra-violet light curtains. In embodiments, each of the plurality of ultra-violet light curtains has at least one first ultra-violet light source positioned along a first side, at least one second ultra-violet light source positioned along a second side opposing the first side, at least one third ultra-violet light source positioned along a third side and at least one fourth ultra-violet light source positioned along a fourth side opposing the third side. In some embodiments, the at least one first, at least one second and at least one third ultra-violet light sources are positioned above the surfaces of the rollers and respectively illuminate first, second and third surfaces of the divest tray while being transported by the third conveyor and the at least one fourth ultra-violet light source transmits ultra-violet light through the spaces to illuminate a fourth surface of the divest tray as it is being transported by the third conveyor.

Referring back to FIGS. 3E and 3F, in some embodiments, a first plurality of nozzles 372 are positioned vertically along or parallel to the tray elevator 350. As the tray 310 leaves the reclaim stations, regions or areas 320 the first plurality of nozzles 372 spray a disinfectant onto the tray 310 as the tray 310 is brought down from the clear conveyor lane 330 onto the return conveyor lane 355 (that lies at a level below the clear conveyor lane 330). Thereafter, as shown in FIG. 3F, tray 310 is transported through the plurality of UV light curtains 360. In some embodiments, the use of the plurality of UV light curtains 360, at this stage, is optional.

In various embodiments, the disinfectant/sanitizer may include alcohol plus quaternary ammonium cation based compounds, aldehydes such as formaldehyde and glutaraldehyde or any other disinfectant (dry or liquid) known in the art.

Referring to FIGS. 3F and 3G, in some embodiments, while being conveyed by the return conveyor lane 355, the tray 310 is passed through a disinfectant bath, using a second plurality of nozzles 374 positioned in the spaces or gaps between the plurality of conveying rollers 358, the second plurality of nozzles 374 being configured to spray a disinfectant onto the tray 310 in order to clean the base 310d of the tray 310. In some embodiments, while being conveyed by the return conveyor lane 355 a plurality of car-wash like rotating brushes 375 are deployed above the return conveyor lane 355 (and along a portion of the return conveyor lane 355 extending from the tray elevator 350 and the exit or entry area of the screening system 304) to clean the top side 310b and inside surfaces of the tray 310. In some embodiments, each of the rotating brushes 375 is coated in a disinfectant. In some embodiments, a drying system 377 is deployed (along a portion of the return conveyor lane 355) beyond the plurality of brushes 375 and prior to the divest stations, regions or areas 315 to remove disinfectant droplets and dry the tray 310. In various embodiments, the drying system 377 includes at least one of dry rotating brushes, infra-red lamps, hot air jets or any other drying means known to persons of ordinary skill in the art.

Thereafter, in some embodiments, the tray 310 is transported through another set of UV light curtains 360 positioned beyond the drying system 377 and prior to the divest stations, regions or areas 315. In some embodiments, the use of the plurality of UV light curtains 360, at this stage (that is beyond the drying system 377 and prior to the divest stations, regions or areas 315), is optional. Thus, a thoroughly disinfected, dried and cleaned tray 310 is returned to the divest stations, regions or areas 315 (FIG. 3H) for use by another passenger.

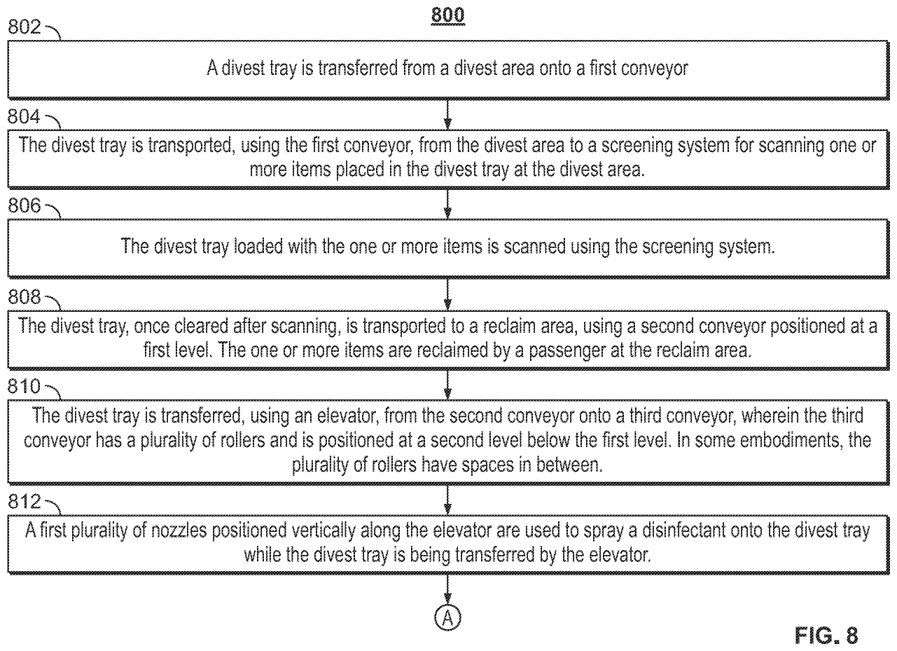

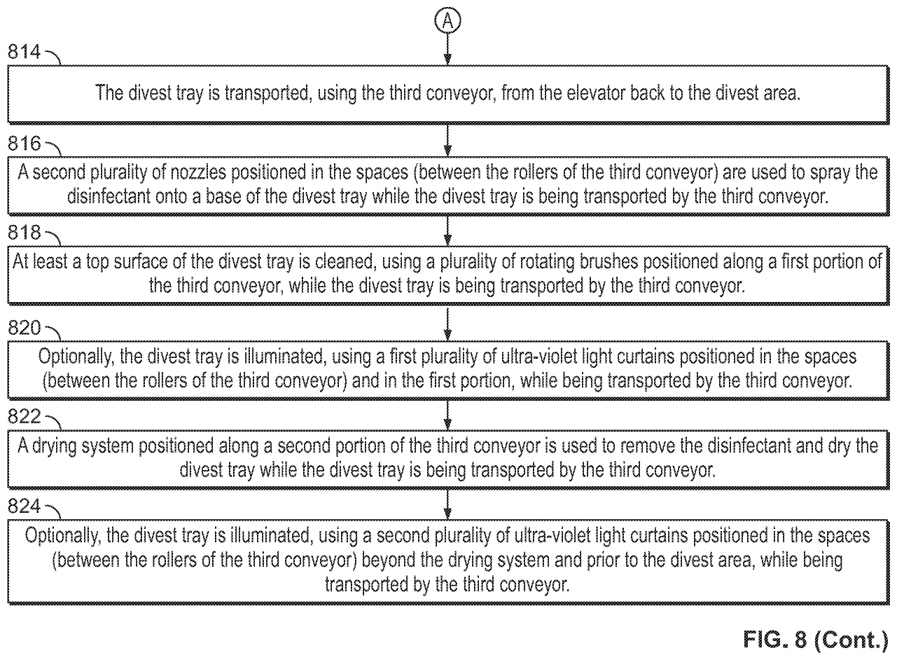

FIG. 8 is a flowchart of a plurality of exemplary steps of a method 800 of sterilizing a divest tray, in accordance with some embodiments of the present specification. In various embodiments, the method 800 is implemented in the checkpoint security system 300 of FIGS. 3A through 3J wherein the system 300 deploys a tray return system.

At step 802, the divest tray is transferred from a divest area onto a first conveyor. A passenger typically divests and places one or more items in the divest tray at the divest area. In some embodiments, the passenger is allowed to place the divest tray onto a divest queuing conveyor, at the divest area, and a tray divert unit enables the tray to be transferred from the divest queuing conveyor onto the first conveyor.

At step 804, the divest tray is transported, using the first conveyor, from the divest area to a screening system for scanning the one or more items placed in the divest tray at the divest area. Thereafter, at step 806, the divest tray loaded with the one or more items is scanned using the screening system.

At step 808, the divest tray, that has been cleared after scanning, is transported, using a second conveyor from the scanning system to a reclaim area, wherein the second conveyor is positioned at a first level. The one or more items are reclaimed by the passenger at the reclaim area. At step 810, the unloaded divest tray is transferred, using an elevator, from the second conveyor onto a third conveyor, wherein the third conveyor has a plurality of rollers and is positioned at a second level below the first level. In some embodiments, the plurality of rollers have spaces in between.

At step 812, a first plurality of nozzles positioned vertically along the elevator are used to spray a disinfectant onto the divest tray while the divest tray is being transferred by the elevator. At step 814, the divest tray is transported, using the third conveyor, from the elevator back to the divest area. At step 816, a second plurality of nozzles positioned in the spaces (between the rollers of the third conveyor) are used to spray the disinfectant onto a base of the divest tray while the divest tray is being transported by the third conveyor.

Thereafter, at step 818, at least a top surface of the divest tray is cleaned, using a plurality of rotating brushes positioned along a first portion of the third conveyor, while the divest tray is being transported by the third conveyor. In some embodiments, the first portion extends from the elevator to the exit area of the screening system.

Optionally, in some embodiments, at step 820, the divest tray is illuminated, using a first plurality of ultra-violet light curtains positioned in the spaces (between the rollers of the third conveyor) and in the first portion, while being transported by the third conveyor. In some embodiments, each of the first plurality of ultra-violet light curtains has at least one first ultra-violet light source positioned along a first side, at least one second ultra-violet light source positioned along a second side opposing the first side, at least one third ultra-violet light source positioned along a third side and at least one fourth ultra-violet light source positioned along a fourth side opposing the third side, wherein the at least one first, second and third ultra-violet light sources are positioned above the surfaces of the rollers and respectively illuminate first, second and third surfaces of the divest tray, and wherein the at least one fourth plurality of ultra-violet light sources transmits ultra-violet light through the spaces to illuminate a fourth surface of the divest tray.

In some embodiments, a plane of each of the first plurality of ultra-violet light curtains lies substantially orthogonal to a direction of movement of the divest tray by the third conveyor.

At step 822, a drying system positioned along a second portion of the third conveyor is used to remove the disinfectant and dry the divest tray while the divest tray is being transported by the third conveyor. In some embodiments, the second portion lays beyond the plurality of rotating brushes and prior to the divest area. In some embodiments, the drying system includes at least one of dry rotating brushes, infra-red lamps, or hot air jets.

Optionally, in some embodiments, at step 824, the divest tray is illuminated, using a second plurality of ultra-violet light curtains positioned in the spaces (between the rollers of the third conveyor) beyond the drying system and prior to the divest area, while being transported by the third conveyor. In some embodiments, each of the second plurality of ultra-violet light curtains has at least one first ultra-violet light source positioned along a first side, at least one second ultra-violet light source positioned along a second side opposing the first side, at least one third ultra-violet light source positioned along a third side and at least one fourth ultra-violet light source positioned along a fourth side opposing the third side, wherein the at least one first, second and third ultra-violet light sources are positioned above the surfaces of the rollers and respectively illuminate first, second and third surfaces of the divest tray, and wherein the at least one fourth ultra-violet light source transmits ultra-violet light through the spaces (between the rollers of the third conveyor) to illuminate a fourth surface of the divest tray.

In some embodiments, a plane of each of the second plurality of ultra-violet light curtains lies substantially orthogonal to a direction of movement of the divest tray by the third conveyor.

Referring now to FIG. 3C, in some embodiments, the screening system 304 has its own dedicated conveyor 380 having an associated conveyor belt 382. The conveyor 380 receives the tray 310 from the input conveyor lane 325 and moves the tray 310 through the inspection tunnel of the system 304 for scanning, in a direction from left to right. In some embodiments, the belt 382 has a first plurality of flaps, spaced apart from each other by a predefined distance and that hinge up from the surface of the belt 382 immediately behind a trailing edge of the tray 310 as the tray 310 is received on the conveyor 380. Each of the first plurality of flaps is configured to catch a tray as it passes through shielding curtains at an entrance of the inspection tunnel of the screening system 304. This prevents the shielding curtains from causing slippage and miss-tracking of the tray.

As the belt 382 loops back in the other direction, from right to left, each of the first plurality of flaps folds back down to lay flush with the surface of the belt 382 in order to minimize snagging on the return path. In embodiments, the first plurality of flaps are hinged up when they get back to the top surface of the conveyor 380 using a mechanical lever (attached to each of the plurality of flaps) and an actuator (such as, for example, a metallic component bolted to a side of the conveyor 380) to enable each of the first plurality of flaps to pop-up.

In some embodiments, the return conveyor lane 355 (FIG. 3G) also includes at least one belt having a second plurality of flaps spaced apart from each other by a predefined distance and hinged up from the top surface of the belt. In some embodiments, the second plurality of flaps are hinged up using a mechanical lever (attached to each of the plurality of flaps) and an actuator (such as, for example, a metallic component bolted to a side of the conveyor 355). The second plurality of flaps assist in pushing a floating tray through the disinfectant bath and/or to prevent the tray from moving or dislodging while being cleaned by the rotating brushes 375 or being dried by the drying system 377. In embodiments, the second plurality of flaps also enables stopping damp trays, following the disinfectant bath, from slipping on the input conveyor lane 325 (FIG. 3C) when loaded with passenger baggage.

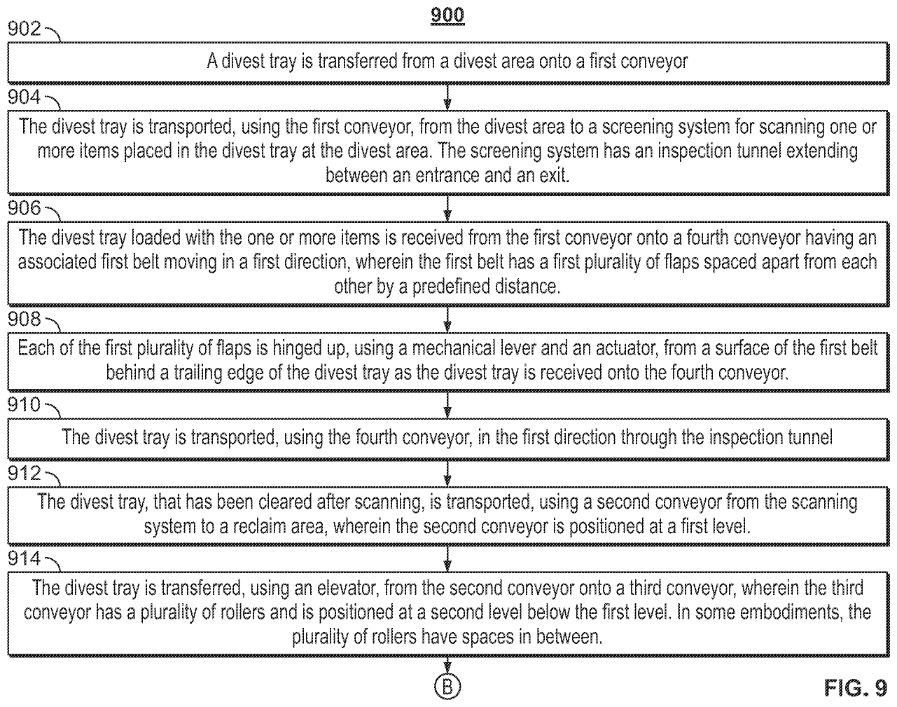

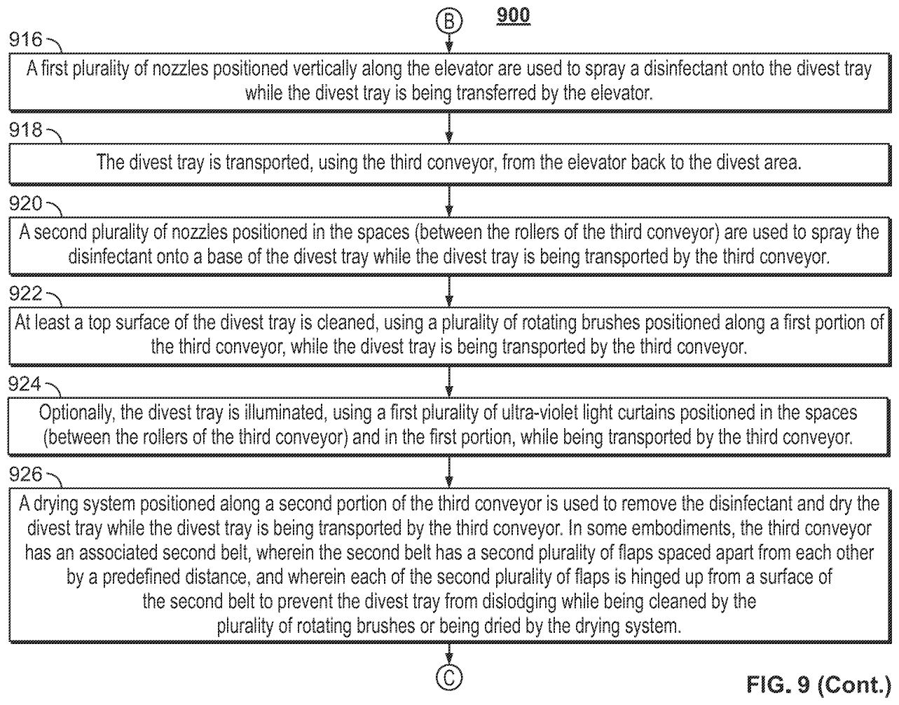

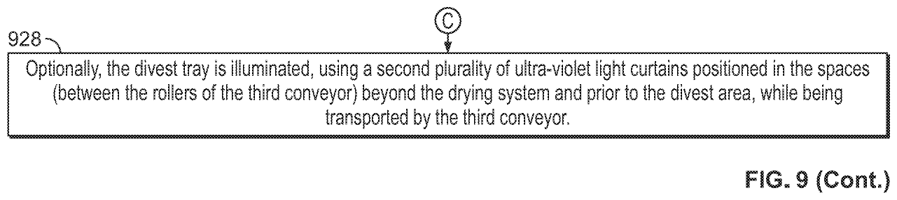

FIG. 9 is a flowchart of a plurality of exemplary steps of a method 900 of sterilizing a divest tray, in accordance with some embodiments of the present specification. In various embodiments, the method 900 is implemented in the checkpoint security system 300 of FIGS. 3A through 3J wherein the system 300 deploys a tray return system.

At step 902, the divest tray is transferred from a divest area onto a first conveyor. A passenger typically divests and places one or more items in the divest tray at the divest area. In some embodiments, the passenger is allowed to place the divest tray onto a divest queuing conveyor, at the divest area, and a tray divert unit enables the tray to be transferred from the divest queuing conveyor onto the first conveyor.

At step 904, the divest tray is transported, using the first conveyor, from the divest area to a screening system for scanning the one or more items placed in the divest tray at the divest area. The screening system has an inspection tunnel extending between an entrance and an exit. At step 906, the divest tray loaded with the one or more items is received from the first conveyor onto a fourth conveyor having an associated first belt moving in a first direction, wherein the first belt has a first plurality of flaps spaced apart from each other by a predefined distance. In some embodiments, the fourth conveyor extends between the entrance and the exit of the screening system.

At step 908, each of the first plurality of flaps is hinged up, using a mechanical lever and an actuator, from a surface of the first belt behind a trailing edge of the divest tray as the divest tray is received onto the fourth conveyor. In embodiments, each of the first plurality of flaps is configured to catch the divest tray as it passes through one or more shielding curtains at the entrance. In some embodiments, each of the first plurality of flaps folds back down to lay flush with the surface as the first belt loops below the fourth conveyor in a second direction opposite to the first direction.

Thereafter, at step 910, the divest tray is transported, using the fourth conveyor, in the first direction through the inspection tunnel.

At step 912, the divest tray, that has been cleared after scanning, is transported, using a second conveyor from the scanning system to a reclaim area, wherein the second conveyor is positioned at a first level. The one or more items are reclaimed by the passenger at the reclaim area. At step 914, the unloaded divest tray is transferred, using an elevator, from the second conveyor onto a third conveyor, wherein the third conveyor has a plurality of rollers and is positioned at a second level below the first level. In some embodiments, the plurality of rollers have spaces in between.

At step 916, a first plurality of nozzles positioned vertically along the elevator are used to spray a disinfectant onto the divest tray while the divest tray is being transferred by the elevator. At step 918, the divest tray is transported, using the third conveyor, from the elevator back to the divest area. At step 920, a second plurality of nozzles positioned in the spaces (between the rollers of the third conveyor) are used to spray the disinfectant onto a base of the divest tray while the divest tray is being transported by the third conveyor.

Thereafter, at step 922, at least a top surface of the divest tray is cleaned, using a plurality of rotating brushes positioned along a first portion of the third conveyor, while the divest tray is being transported by the third conveyor. In some embodiments, the first portion extends from the elevator to the exit area of the screening system.

Optionally, in some embodiments, at step 924, the divest tray is illuminated, using a first plurality of ultra-violet light curtains positioned in the spaces (between the rollers of the third conveyor) and in the first portion, while being transported by the third conveyor. In some embodiments, each of the first plurality of ultra-violet light curtains has at least one first ultra-violet light source positioned along a first side, at least one second ultra-violet light source positioned along a second side opposing the first side, at least one third ultra-violet light source positioned along a third side and at least one fourth ultra-violet light source positioned along a fourth side opposing the third side, wherein the at least one first, second and third ultra-violet light sources are positioned above the surfaces of the rollers and respectively illuminate first, second and third surfaces of the divest tray, and wherein the at least one fourth ultra-violet light source transmits ultra-violet light through the spaces to illuminate a fourth surface of the divest tray.

In some embodiments, a plane of each of the first plurality of ultra-violet light curtains lies substantially orthogonal to a direction of movement of the divest tray by the third conveyor.

At step 926, a drying system positioned along a second portion of the third conveyor is used to remove the disinfectant and dry the divest tray while the divest tray is being transported by the third conveyor. In some embodiments, the second portion lays beyond the plurality of rotating brushes and prior to the divest area. In some embodiments, the drying system includes at least one of dry rotating brushes, infra-red lamps, or hot air jets.

In some embodiments, the third conveyor has an associated second belt, wherein the second belt has a second plurality of flaps spaced apart from each other by a predefined distance, and wherein each of the second plurality of flaps is hinged up from a surface of the second belt to prevent the divest tray from dislodging while being cleaned by the plurality of rotating brushes or being dried by the drying system.

Optionally, in some embodiments, at step 928, the divest tray is illuminated, using a second plurality of ultra-violet light curtains positioned in the spaces (between the rollers of the third conveyor) beyond the drying system and prior to the divest area, while being transported by the third conveyor. In some embodiments, each of the second plurality of ultra-violet light curtains has at least one first ultra-violet light source positioned along a first side, at least one second ultra-violet light source positioned along a second side opposing the first side, at least one third ultra-violet light source positioned along a third side and at least one fourth ultra-violet light source positioned along a fourth side opposing the third side, wherein the at least one first, second and third ultra-violet light sources are positioned above the surfaces of the rollers and respectively illuminate first, second and third surfaces of the divest tray, and wherein the at least one fourth ultra-violet light sources transmits ultra-violet light through the spaces (between the rollers of the third conveyor) to illuminate a fourth surface of the divest tray.

In some embodiments, a plane of each of the second plurality of ultra-violet light curtains lies substantially orthogonal to a direction of movement of the divest tray by the third conveyor.

Referring back to FIG. 3A, in some embodiments, the input conveyor lane 325, responsible for conveying trays into the screening system 304, and the clear conveyor lane 330, responsible for conveying trays out of the screening system 304, constitute a single main conveyor lane of the tray return system 305 and that the qualifiers ‘input’ and ‘clear’ are indicative of the operating function of these conveyors. Also, all conveyors (conveyors 325, 330, 335, and 337) of the tray return system 305 comprise a plurality of main rollers, covered by a rubber, plastic, or other material, that rotate to impart linear velocity to the material coverings and, therefore to the trays mounted thereon, in a plurality of tray or inspection slots.

In embodiments, the tray return system 305 has a fixed length for each of the plurality of tray slots (or inspection slots). In some embodiments, the length of a tray slot is in a range of 400 to 1200 mm, preferably in a range of 600 to 1000 mm and more preferably 800 mm. Oversized items such as a musical instrument or a walking stick, for example, are accommodated over multiple tray slot lengths, such as for example, 2 tray slot lengths for a guitar or similar item.

In embodiments, each tray has a length ‘l’ and a width ‘w’ with a minimum gap ‘g’ between adjacent trays. In some embodiments, the length ‘l’ is in a range of 350 to 1150 mm, preferably in a range of 550 to 950 mm and more preferably 750 mm, while the gap ‘g’ is in a range of 30 to 80 mm, preferably 50 mm. In some embodiments, the width ‘w’ is up to 550 mm to allow scanning of objects (held in the trays) in X-ray baggage screening systems that have at least 600 mm wide inspection tunnels.

In some embodiments, the tray return system 305 is capable of operating at speeds that can vary between 0.1 m/s and 0.5 m/s. The operating speed may vary at each position within the system 300 depending on operational conditions and in order to maximize tray throughput. In some embodiments, different conveyors 325, 330, 335, and 337 of the tray return system 305, operate at different speeds and a tray positioned at any of the conveyors may be decelerated or accelerated independently within the system 305. For example, if the clear lane 330 is empty, a tray may be conveyed at a high speed along the conveyor 330 to the reclaim stations 320. Alternatively, if the clear lane 330 is becoming full, the input conveyor 325 and the conveyor of the scanning system 304 may be slowed down to give more time for the lane 330 to clear without having to stop the divest or scanning process. In various embodiments, the conveyors of the system 305 do not stop and their direction of movement is not reversed, during operation of the system.

In various embodiments, at the divest stations 315 and reclaim stations 320 the trays are in a stationary position, enabling a passenger at a divest station 315 to pull a tray out of a tray slot, place it on top of the divest station conveyor 337d, and fill the tray while the tray stays in the same location. Once done, the passenger pushes the filled tray onto the tray divert unit conveyor element 340d. The tray return system 300 then moves the tray onto conveyor 325 at an appropriate time based on the clearance of the tray divert units 340 and the reclaim stations 320. Similarly, the tray that arrives at the reclaim stations remain in a stationary position until the passenger lifts it to reclaim his/her belongings.

In some embodiments, each sub-system or component (such as, the divest stations 315, reclaim stations 320, divert units 340, input conveyor 325, clear conveyor lane 330, divert conveyor lane 335, divest and reclaim queuing conveyor lanes 337) of the tray return system 305 has at least one dedicated local microprocessor (such as a CANbus controller node) responsible for managing the operation of various elements, units or devices of the associated sub-system or component.

### Exemplary Screening Systems

Referring to FIGS. 1A and 3A, in some embodiments, the screening system 104/304 is a real-time tomography (RTT) system having a substantially circular locus of source points used to scan a scanning volume. In another embodiment, the screening system 104/304 is a real-time tomography (RTT) system having a substantially rectangular or non-circular locus of source points used to scan a scanning volume. In an embodiment, a plurality of X-ray source points is arranged in a non-circular or substantially rectangular geometry around the scanning volume. Due to the non-circular geometry of the X-ray source points, the RTT system is cost effective, has a smaller footprint and may be operated using regular line voltage to supply power to the high voltage power supply, which is then used to provide power to the X-ray source.

In various embodiments, the X-ray sources emit fan beams which have different beam angles based on the location of the X-ray source points with respect to an imaging volume.

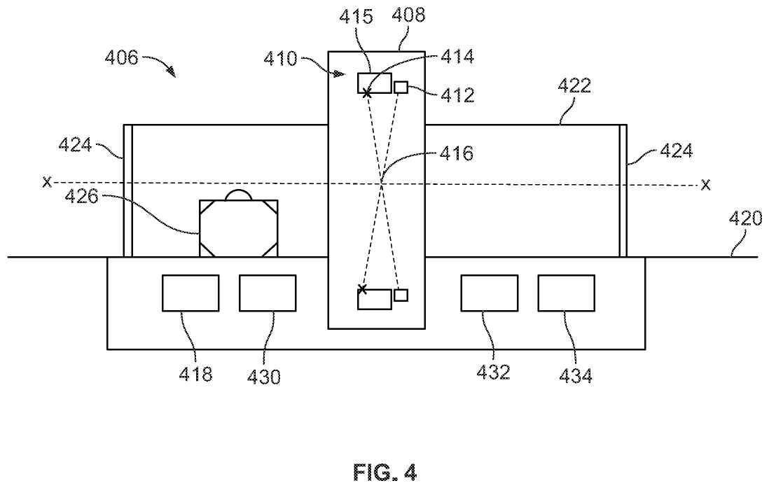

FIG. 4 illustrates a conventional RTT screening system having a circular locus of source points. Referring to FIG. 4, a concourse parcel and/or baggage scanning system 406 comprises a scanning unit 408 which includes a multi-focus X-ray source 410 and X-ray detector array 412. The source 410 comprises a large number of source points 414 positioned in respective, spaced locations on an emitter 415, and arranged in a full 360 degree circular array about the X-X axis of the system (which is parallel to the conveyor belt 420). It will be appreciated that curved arrays covering less than the full 360 degree angle can also be used. The source 410 can be controlled to produce X-rays from each of the source points 414 in each of the source units individually whereby X-rays from each source point 414 are directed inwards through the scanning region 416 within the circular source 410. The source 410 is controlled by a control unit 418 which controls the applied electrical potentials (to the grid structures) and hence controls the emission of X-rays from each of the source points 414.

The multi-focus X-ray source 410 allows the electronic control circuit 418 to be used to select which of the many individual X-ray source points 414 within the multi-focus X-ray source is active at any moment in time. Hence, by electronically scanning the multi-focus X-ray tube, X-ray source virtual “motion” is created with no actual physical movement of mechanical parts. In this case, the angular velocity of source rotation can be increased to levels that simply cannot be achieved when using conventional rotating X-ray tube assemblies. This rapid rotational scanning translates into an equivalently speeded up data acquisition process and, as a result, fast image reconstruction.

The detector array 412 is also circular and arranged around the axis X-X in a position that is slightly offset in the axial direction from the source 410. The source 410 is arranged to direct the X-rays it produces through the scanning region 416 towards the detector array 412 on the opposite side of the scanning region. The paths 417 of the X-ray beams therefore pass through the scanning region 416 in a direction that is substantially, or almost, perpendicular to the scanner axis X-X, crossing each other near to the axis. The volume of the scanning region that is scanned and imaged is therefore in the form of a thin slice perpendicular to the scanner axis X-X. The source is scanned so that each source point emits X-rays for a respective period, the emitting periods being arranged in a predetermined order. As each source point 414 emits X-rays, the signals from the detectors 412, which are dependent on the intensity of the X-rays incident on the detector, are produced, and the intensity data that the signals provide are recorded in a memory. When the source has completed its scan the detector signals can be processed to form an image of the scanned volume.

A conveyor belt 420 moves through the imaging volume, from left to right, as seen in FIG. 4, parallel to the axis X-X of the scanner. X-ray scatter shields 422 are located around the conveyor belt 420 upstream and downstream of the main X-ray system to prevent operator dose due to scattered X-rays. The X-ray scatter shields 422 include lead rubber strip curtains 424 at the open ends of the system such that the item 426 under inspection is conveyed through one curtain on entering the inspection region and another curtain upon leaving the inspection region. In the integrated system shown, the main electronic control system 418, a processing system 430, a power supply 432 and cooling racks 434 are shown mounted underneath the conveyor 420. The conveyor 420 is arranged to be operated normally with a continuous scanning movement at constant conveyor speed, and typically has a carbon-fiber frame assembly within the imaging volume.

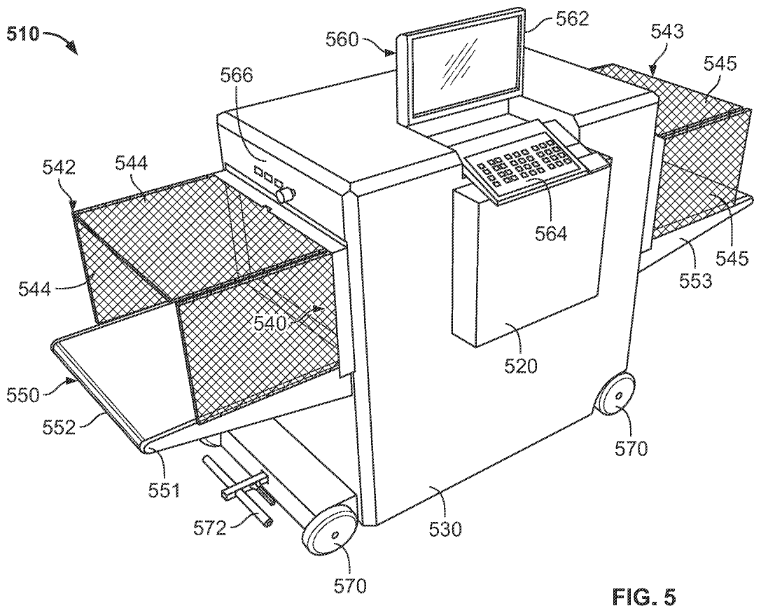

With reference FIGS. 1A and 3A, in some embodiments, the screening system 104/304 is an X-ray security inspection machine 500 of FIG. 5. Referring to FIG. 5, a photodetector array module 520 has been extended to its deployed position and projects from the X-ray machine's housing 530. The extent of projection of the photodetector array module 520 is typically between 60 mm and 100 mm, but preferably the module extends by approximately 80 mm from the housing.

Contained within the housing 530 are the necessary components for providing an X-ray security inspection, including an X-ray generator, processing and control electronics, power and control cables, a computer processor and memory storing suitable operational software, and X-ray shielding material, as will be understood by a person of ordinary skill in the art.

Above the X-ray generator (not shown) within the housing 530 and below the upper surface of the photodetector array module 520, the housing defines a tunnel 540, having a substantially rectangular tunnel opening 541. Items (such as, for example, parcels and/or baggage) to undergo X-ray inspection in the machine 510 pass through the tunnel 540 for irradiation by X-ray photons. Those photons which are transmitted through the item under inspection are then detected. The tunnel opening 541 has standard dimensions of 640 mm wide by 430 mm high, in this embodiment.

The tunnel 540 passes through the housing 530 from an input side 542 to an output side 543. In order to help define a maximum item size which may be inspected with the machine 510, a respective plurality of tunnel wall panels 544, 545 are disposed around the tunnel openings at the input and output sides 542, 543. The panels are preferably made of a transparent plastics material, such as polycarbonate, Plexiglas™ or Perspex™, to facilitate observation of items passing into and out of the tunnel 540.

A conveyor system 550 transports items for inspection from the input side 542 of the tunnel 540 to its output side 543. In the embodiment shown in FIG. 5, the conveyor system 550 comprises three separate conveyors: an input conveyor 551 having an associated conveyor belt 552; an output conveyor 553 having an associated conveyor belt 554; and an intermediate conveyor (not shown) located within the tunnel 540 and having its own associated conveyor belt (also not shown). Other arrangements of the conveyor system will be readily apparent to a person of ordinary skill in the art.

The X-ray security inspection machine 510 incorporates a control console 560, comprising a monitor 562 and a keypad/mouse pad 564, by means of which an operator may control the machine. The control console 560 is stowable, so that it does not contribute to the overall width of the machine 510, when in its stowed configuration.

Initial activation and subsequent deactivation of the machine 510 are achieved by means of activation control switches 566, which are accommodated on the housing 530, preferably not on either lateral sides of the machine, so as not to increase the width profile of the machine. The activation controls 566 may include a key switch to ensure operation only by authorized personnel, or the like.

The X-ray security inspection machine 510 is provided with a set of wheels or castors 570, on which the machine is supported and by means of which the machine may be moved. The wheels 570 may be standard nylon wheels, conventionally used. However, nylon wheels tend to be relatively hard which makes movement of the machine over certain surfaces, especially uneven surfaces, somewhat difficult. Preferably, the wheels are provided by rubber castors, which are capable of conforming more readily to surface unevenness and reducing the impact on the machine when encountering such surfaces. This specification of wheel also reduces vibration of the machine during transportation. In order to facilitate such movement, a steering and braking handle 572 is provided. The steering and braking handle 572 co-operates with the wheels 570, at least to provide a braking mechanism if not also to provide a steering mechanism for changing direction of the machine. In the deployed configuration of the machine 510 ready for use, the steering and braking handle 572 is itself stowed away beneath the machine, as shown in FIG. 5.

The above examples are merely illustrative of the many applications of the system and method of present specification. Although only a few embodiments of the present specification have been described herein, it should be understood that the present specification might be embodied in many other specific forms without departing from the spirit or scope of the specification. Therefore, the present examples and embodiments are to be considered as illustrative and not restrictive, and the specification may be modified within the scope of the appended claims.

1. A screening system for inspecting an object and sterilizing the object, comprising:
a scanner having a housing defining an inspection volume, a radiation source coupled to the inspection volume, and a detector array coupled to the inspection volume, wherein the housing comprises an upstream side configured to allow the object to enter the inspection volume and a downstream side configured to allow the object to exit the inspection volume;
an entry shroud having a first end and a second end, wherein the first end is coupled to the upstream side and wherein the entry shroud comprises a first frame extending between the first end and the second end;
an exit shroud having a third end and a fourth end, wherein the third end is coupled to the downstream side and wherein the exit shroud comprises a second frame extending between the third end and the fourth end;
a first plurality of ultraviolet light curtains coupled to the entry shroud, wherein the first plurality of ultraviolet light curtains are positioned between the first end and the second end; and
a first conveyor extending at least from the second end to the first end of the entry shroud and a second conveyor extending at least from the third end to the fourth end of the exit shroud, wherein the first conveyor is configured to transport the object from the second end through the first plurality of ultra-violet light curtains to the inspection volume and the second conveyor is configured to transport the object from the inspection volume after scanning to the fourth end.

2. The scanning system of claim 1, wherein each of the first conveyor and the second conveyor include at least two belts and wherein the at least two belts having at least one split space between them.

3. The scanning system of claim 1, wherein each of the first plurality of ultra-violet light curtains comprises a first ultra-violet light source positioned along a first side of the first frame, a second ultra-violet light source positioned along a second side of the first frame, wherein the second side opposes the first side, a third ultra-violet light source positioned along a third side of the first frame and a fourth ultra-violet light source positioned along a fourth side of the first frame, wherein the third side opposes the fourth side.

4. The scanning system of claim 3, wherein the first ultra-violet light source, the second ultra-violet light source and the third ultra-violet light source are positioned above belt surfaces of the first conveyor and are configured to illuminate a first surface, a second surface and a third surface, respectively, of the object while being transported by the first conveyor.

5. The scanning system of claim 4, wherein the fourth ultra-violet light source is positioned below the belt surfaces to direct ultra-violet light through a space in the belt surfaces to thereby illuminate a fourth surface of the object.

6. The scanning system of claim 1, wherein the first frame lies in a plane that is substantially orthogonal to a direction of movement of the object by the first conveyor.

7. The scanning system of claim 1, wherein each of the first plurality of ultra-violet light curtains uses light sources emitting ultra-violet radiation in a wavelength range of 280-100 nm or photon energy in a range of 4.43-12.4 eV.

8. The scanning system of claim 1, further comprising a lead lined cover coupled to at least one of the first frame and the second frame.

9. The scanning system of claim 1, wherein the entry shroud comprises a first portion extending from the second end, wherein the first portion is coupled to a second portion, wherein the second portion is coupled to a third portion, wherein the third portion is coupled to the first end, and wherein the first portion and third portion are positioned at a right angle to each other.

10. The scanning system of claim 1, wherein the entry shroud comprises a first portion extending from the second end, wherein the first portion is coupled to a second portion, wherein the second portion is coupled to a third portion, wherein the third portion is coupled to the first end, wherein a longitudinal axis of the first portion and a longitudinal axis of the third portion are parallel to each other, and wherein the second portion is skewed at an angle of 45 degrees relative to the longitudinal axes of both the first portion and the third portion.

11. The scanning system of claim 1, wherein the entry shroud comprises a first portion extending from the second end, wherein the first portion is coupled to a second portion, wherein the second portion is coupled to a third portion, wherein the third portion is coupled to the first end, and wherein the first portion and third portion are configured such that a longitudinal axis of the first portion and a longitudinal axis of the third portion are neither parallel nor orthogonal to each other.

12. The scanning system of claim 1, wherein the object is a parcel or a baggage.

13. A method of sterilizing an item to be scanned by a scanning system having an upstream side and a downstream side adapted to allow the item to enter and exit, respectively, the scanning system, wherein the upstream side is coupled to an entry shroud having a first end and a second end, wherein the downstream side is coupled to an exit shroud having a third end and a fourth end, wherein a first plurality of ultra-violet light curtains are positioned within the entry shroud between the first end and the second end, the method comprising:
placing the item on a first conveyor at the second end, wherein the first conveyor extends at least from the second end to the first end within the entry shroud;
transporting, using the first conveyor, the item from the second end to the scanning system through said first plurality of ultra-violet light curtains, wherein each of said first plurality of ultra-violet light curtains has a least a first ultra-violet light source positioned along a first side, at least a second ultra-violet light source positioned along a second side opposing the first side, at least a third ultra-violet light source positioned along a third side and at least a fourth ultra-violet light source positioned along a fourth side opposing the third side; and
illuminating first, second, third and fourth surfaces of the item by the at least one first, at least one second, at least one third and at least one fourth ultra-violet light sources while the item is being transported by the first conveyor.

14. The method of claim 13, further comprising:
scanning the item using the scanning system; and
transporting, using a second conveyor, the item from the scanning system to said fourth end, wherein said second conveyor extends at least from the third end to the fourth end within the exit shroud.

15. The method of claim 14, wherein a second plurality of ultra-violet light curtains is positioned within the exit shroud between the third end and the fourth end.

16. The method of claim 15, wherein each of the second plurality of ultra-violet light curtains has at least one first ultra-violet light source positioned along a first side, at least one second ultra-violet light source positioned along a second side opposing the first side, at least one third ultra-violet light source positioned along a third side and at least one fourth ultra-violet light source positioned along a fourth side opposing the third side.

17. The method of claim 16, further comprising:
illuminating first, second, third and fourth surfaces of the item by the at least one first, at least one second, at least one third and at least one fourth ultra-violet light source of each of said second plurality of ultra-violet light curtains while the item is being transported by the second conveyor.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)
